(19) United States
(12) Patent Application Publication
(10) Pub. No.: US 2005/0191349 A1
(43) Pub. Date: Sep. 1, 2005

(54) Galantamine formulations

(76) Inventors: Garth Boehm, Westfield, NJ (US); Josephine Dundon, Fanwood, NJ (US)

Correspondence Address: CANTOR COLBURN, LLP, 55 GRIFFIN ROAD SOUTH, BLOOMFIELD, CT, 06002, US

(21) Appl. No.: 11/001,712

(22) Filed: Dec. 1, 2004

Related U.S. Application Data
(60) Provisional application No. 60/533,571, filed on Dec. 31, 2003.

(51) Int. Cl. A61K 31/55; A61K 31/473; A61K 31/4178; A61K 9/20

(52) U.S. Cl. 424/464; 514/214.03; 514/397; 514/297

(57) ABSTRACT

Galantamine formulations, including sustained-release and fast dissolve formulations, are described.

### CROSS-REFERENCE TO RELATED APPLICATIONS

This application claims the benefit of U.S. Provisional Application No. 60/533,571, filed Dec. 31, 2003, which is incorporated by reference herein in its entirety.

### BACKGROUND

Galantamine (I) ((4aS,6R,8aS)-4a,5,9,10,11,12-hexahydro-3-methoxy-11-methyl6H-benzofuro[3a,3,2-ef][2]benzazepin-6-ol) is a known reversible, 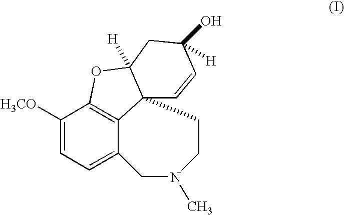
competitive acetylcholinesterase inhibitor. The compound has been isolated from the bulbs of the Caucasian snowdrops Galantanus woronowi in addition to the common snowdrop Galanthus Nivalis.

Galantamine and its salts, have been employed as a pharmaceutically active agent in the treatment of a variety of disorders, including mania, alcoholism, nicotine dependence, and Alzheimer's disease. In particular, galantamine hydrobromide has been used for the treatment of Alzheimer's disease and is currently formulated as film-coated tablets of 4 milligram (mg), 8 mg, and 12 mg doses for twice a day oral administration under the trade name REMINYL.

As an acetylcholinesterase inhibitor, galantamine is known to be active at nicotinic receptor sites, but not on muscarinic receptor sites. It is capable of passing the blood-brain barrier in humans, and presents no severe side effects in therapeutically effective dosages. Although no severe side effects are found, when first dosed, patients may experience the occurrence of numerous side effects, which affect the patients' tolerability of the drug. Side effects, such as nausea or vomiting and headaches, often occur when the drug is introduced at high doses. An initial therapeutic regimen often starts with first introducing galantamine at low doses for several weeks followed by the gradual increase to the optimal active dose for the patient. When the regular dosing of galantamine is interrupted for two or more days, it is recommended to commence dosing at the lowest levels as continuation at the doses prior to the interruption are generally not well tolerated by the patient.

Previously described formulations of this active agent have certain properties that are not ideal in all situations.

Manufacturing problems associated with fast dissolving galantamine hydrobromide tablets has been disclosed in the art. It was determined that using either lactose anhydrous or lactose monohydrate as a diluent, and either powdered cellulose or microcrystalline cellulose as a disintegrant resulted in segregation of the tablet excipients, resulting in tablets having a variable composition. Using a spray-dried mixture of lactose monohydrate and microcrystalline cellulose (75:25) as the diluent in combination with a disintegrant having a large coefficient of expansion was describe as providing a formulation that avoided the segragation problems while at the same time resulting in a targeted dissolution profile. Although one formulation has been developed for fast dissolving galantamine hydrobromide tablet, there exists a continuing need for additional formulations, which can be easily processed, provide product consistency, and compliance with product specifications.

Controlled-release galantamine compositions are known (see, for example, WO 00/38686). Such compositions include particles containing galantamine wherein the particles are coated with a release rate controlling membrane coating. Improvements in the area of controlled-release formulations may provide improved pharmacokinetic and/or dissolution profiles not yet achieved by known formulations.

As galantamine is used to treat Alzheimer's disease and other dementias, usually for elderly patients, tablet formulations may result in problems of administration such as difficulty or inability of swallowing. Solutions or suspensions of galantamine have an unpleasant taste. Therefore, formulations that are easy to swallow as well as pleasant to the palate are thus desirable. Known formulations for taste masking include the addition of a sweetening agent. This formulation has its drawback as particles of the active agent may remain in the mouth after the initial ingestion of the formulation only later to be detected by the patient as an unpleasant taste. Thus, new taste masked formulations are needed as well as easily administered formulations, such as chewable tablets, sprinkle forms, liquid formulations, taste-masked formulations, and fast dissolve tablets.

The present invention addresses these and other needs for improved galantamine dosage forms, particularly controlled-release and sustained-release dosage forms.

### SUMMARY OF THE INVENTION

In one embodiment, a dosage formulation comprises galantamine or a pharmaceutically acceptable salt thereof, and a pharmaceutically acceptable carrier, wherein the carrier is substantially free of a spray dried mixture of lactose monohydrate and microcrystalline cellulose; and wherein the formulation exhibits a dissolution profile such that after 0.5 hour at least about 80% of the galantamine or galantamine salt is released after combining the dosage formulation with 500 ml of purified water at 37° C. in Apparatus 2 (USP, <711 > Dissolution, paddle, 50 rpm).

In another embodiment, a dosage formulation comprises galantamine or a pharmaceutically acceptable salt thereof, and a pharmaceutically acceptable carrier; and wherein the formulation exhibits a dissolution profile such that after 0.5 hour less than about 75% of the galantamine or galantamine salt is released after combining the dosage formulation with 500 ml of purified water at 37° C. in Apparatus 2 (USP, <711 > Dissolution, paddle, 50 rpm).

In yet another embodiment, a sustained-release formulation comprises galantamine or a pharmaceutically acceptable salt thereof; and a release-retarding material, wherein the release-retarding material is an acrylate polymer, wax, modified cellulose, shellac, zein, hydrogenated vegetable oil, hydrogenated castor oil, or combinations comprising at least one of the foregoing release-retarding materials, wherein the formulation exhibits a dissolution profile such that less than about 18% of the galantamine is released in 1 hour, and less than about 80% of the galantamine is released in 10 hours after combining the formulation with a dissolution medium at 37° C. in Apparatus 2 (USP, <711> Dissolution, paddle, 50 rpm).

In yet another embodiment, an oral dosage formulation comprises galantamine or a pharmaceutically acceptable salt thereof in controlled-release form, wherein the formulation provides a first maximum plasma concentration of the galantamine (Cₘₐₓ₁) between 0 hours and about 12 hours after administration, and a second maximum plasma concentration of the galantamine (Cₘₐₓ₂) between about 12 hours and about 24 hours after administration at steady-state.

In yet another embodiment, a sustained-release oral dosage formulation comprises a first subunit wherein the first subunit comprises a galantamine or a pharmaceutically acceptable salt thereof, and a first release-retarding material; and a second subunit, wherein the second subunit comprises galantamine or a pharmaceutically acceptable salt thereof, and a second release-retarding material, wherein the first and second release-retarding material can be the same or different, and wherein the dosage formulation, at steady-state, provides a maximum galantamine plasma concentration (Cₘₐₓ) and an galantamine plasma concentration at about 24 hours after administration (C₂₄), wherein the ratio of Cₘₐₓ to C₂₄ is less than about 4:1.

In another embodiment, a dosage formulation comprises a pharmaceutically effective amount of galantamine or a pharmaceutically acceptable salt thereof; and an excipient, wherein the dosage formulation exhibits a dissolution profile such that less than about 18% of the galantamine or galantamine salt is released in 1 hour, and less than about 80% of the galantamine or galantamine salt is released in 10 hours after combining the dosage formulation with USP buffer pH 6.8 at 37° C. in an Apparatus 2 (USP<71 1> Dissolution, paddle, 50 rpm).

In an embodiment, a dosage formulation comprises a pharmaceutically effective amount of galantamine or a pharmaceutically acceptable salt thereof; and a pharmaceutically acceptable excipient, wherein the dosage formulation exhibits a dissolution profile such that after 10 hours less than about 80% of the galantamine or galantamine salt is released after combining the dosage formulation with USP buffer pH 6.8 at 37° C. in an Apparatus 2 (USP, <711> Dissolution, paddle, 50 rpm).

In another embodiment, a dosage formulation comprises a pharmaceutically effective amount of galantamine or a pharmaceutically acceptable salt thereof; and a pharmaceutically acceptable excipient, wherein the dosage formulation exhibits a dissolution profile such that after 1 hour about 5 to about 15% of the galantamine or galantamine salt is released, after 2 hours about 10 to about 25% of galantamine or galantamine salt is released, after 4 hours about 15 to about 35% of the galantamine or galantamine salt is released, and after 8 hours about 25 to about 50% of galantamine or galantamine salt is released.

In another embodiment, a dosage formulation, comprises galantamine or a pharmaceutically acceptable salt thereof; and a pharmaceutically acceptable excipient; and wherein the dosage formulation exhibits a dissolution profile such that at 5 minutes about 0 to about 20% of the galantamine or galantamine salt is released, at 15 minutes about 10 to about 80% of galantamine or galantamine salt is released, at 30 minutes about 20 to about 95% of the galantamine or galantamine salt is released, at 45 minutes about 30 to about 95% of galantamine or galantamine salt is released, and at 60 minutes about 40 to about 95% of galantamine or galantamine salt is released after combining the dosage formulation with 500 ml of an aqueous buffer solution (USP, pH 4.5) at 37° C. in Apparatus 2 (USP, <711 > Dissolution, paddle, 50 rpm).

In still another embodiment, a dosage formulation comprises galantamine or a pharmaceutically acceptable salt thereof; and a pharmaceutically acceptable excipient; and wherein the dosage formulation exhibits a dissolution profile such that at 5 minutes about 0 to about 4% of the galantamine or galantamine salt is released, at 15 minutes about 10 to about 40% of galantamine or galantamine salt is released, at 30 minutes about 20 to about 75% of the galantamine or galantamine salt is released, at 45 minutes about 30 to about 85% of galantamine or galantamine salt is released, and at 60 minutes about 40 to about 85% of galantamine or galantamine salt is released after combining the dosage formulation with 500 ml of an aqueous buffer solution (USP, pH 6.5) at 37° C. in Apparatus 2 (USP, <711 > Dissolution, paddle, 50 rpm).

In another embodiment, a dosage formulation comprises galantamine or a pharmaceutically acceptable salt thereof; and a pharmaceutically acceptable excipient; and wherein the dosage formulation exhibits a dissolution profile such that at 5 minutes about 0 to about 10% of the galantamine or galantamine salt is released, at 15 minutes about 10 to about 35% of galantamine or galantamine salt is released, at 30 minutes about 20 to about 60% of the galantamine or galantamine salt is released, at 45 minutes about 30 to about 80% of galantamine or galantamine salt is released, and at 60 minutes about 40 to about 85% of galantamine or galantamine salt is released after combining the dosage formulation with 500 ml of an aqueous buffer solution (USP, pH 7.5) at 37° C. in Apparatus 2 (USP, <711 > Dissolution, paddle, 50 rpm).

In still another embodiment, a dosage formulation comprises galantamine or a pharmaceutically acceptable salt thereof; and a pharmaceutically acceptable excipient; and wherein the dosage formulation exhibits a dissolution profile such that at 5 minutes about 0 to about 40% of the galantamine or galantamine salt is released, at 15 minutes about 10 to about 90% of galantamine or galantamine salt is released, at 30 minutes about 20 to about 95% of the galantamine or galantamine salt is released, at 45 minutes about 30 to about 98% of galantamine or galantamine salt is released, and at 60 minutes about 40 to about 98% of galantamine or galantamine salt is released after combining the dosage formulation with 500 ml of 0.1N HCl at 37° C. in Apparatus 2 (USP, <711 > Dissolution, paddle, 50 rpm).

A controlled-release formulation comprises particles comprising galantamine or a pharmaceutically acceptable salt thereof reversibly adsorbed onto a cationic ion exchange material, wherein the particles are coated with a polymeric coating material.

These and other advantages of the invention, as well as additional inventive features, will be apparent from the description of the invention provided herein.

### DETAILED DESCRIPTION OF THE INVENTION

Galantamine may be delivered using formulations described herein that provide desirable properties and advantages unavailable in the art. In one embodiment, a dosage formulation comprises galantamine or a pharmaceutically acceptable salt thereof; and a pharmaceutically acceptable carrier, wherein the carrier is substantially free of a spray dried mixture of lactose monohydrate and microcrystalline cellulose; and wherein the formulation exhibits a dissolution profile such that after 0.5 hour at least about 80% of the galantamine or galantamine salt is released in 500 ml of purified water at 37° C. in Apparatus 2 (USP, <711 > Dissolution, paddle, 50 rpm). This formulation provides a quick dissolving galantamine formulation that is easily processed into tablets.

Another embodiment provides controlled-release formulations, such as longer acting formulations that can be administered once daily or even less frequently, which is particularly desirable for this active agent. Controlled-release formulations of this active agent before significant plasma levels of the active agent are achieved provide many inherent therapeutic benefits that are not achieved with corresponding short acting, immediate-release preparations. By providing controlled-release, it is easier to provide steady-state blood levels.

Additionally patient compliance with the dosing regimen, which is problematic among Alzheimer's patients, is improved with dosage formulations that can be administered less frequently.

In certain circumstances controlled-release or pulse delayed-release formulations of the active agent are desirable to reduce the cholenergic side affects associated with the use of galantamine, such as gastric irritation. Thus controlled-release formulations, or pulse delayed-release formulation, with initial low levels of active agent introduced into the plasma with a time-delay, avoiding an initial burst of the active agent helps to reduce the acute cholinergic effects of galantamine.

Another issue is that the current tablet formulations may be inadequate for juvenile and elderly patients who require dosage forms that are easy to swallow. Easily administered formulations, such as chewable tablets, sprinkle forms, liquid formulations, taste-masked formulations, and fast dissolve tablets are thus desirable.

Chemical Description and Terminology

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “active agent” is meant to include solvates (including hydrates) of the free compound or salt, crystalline and non-crystalline forms, as well as various polymorphs. Unless otherwise specified, the term “active agent” is used herein to indicate galantamine or a pharmaceutically acceptable salt thereof. For example, an active agent can include all optical isomers of the compound and all pharmaceutically acceptable salts thereof either alone or in combination.

“Pharmaceutically acceptable salts” includes derivatives of the disclosed compounds, wherein the parent compound is modified by making non-toxic acid addition or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like, and combinations comprising one or more of the foregoing salts. A particularly preferred salt of galantamine is galantamine hydrobromide.

By “water-soluble” active agent is meant an active agent, including galantamine hydrobromide, and other active agents that may be used in combination with active agent that are at least slightly water-soluble (for example, about 1 to about 10 mg/ml at 25° C.). Preferably, all active agents are moderately water-soluble (for example, less than about 100 mg/ml at 25° C.), or highly water-soluble (for example, greater than about 100 mg/ml at 25° C.).

By “water-insoluble” or “poorly soluble” active agent, it is meant an agent having a water solubility of less than 1 mg/ml, and in some cases even less than 0.1 mg/ml.

By “oral dosage form” is meant to include a unit dosage form prescribed or intended for oral administration. An oral dosage form may or may not comprise a plurality of subunits such as, for example, microcapsules or microtablets, packaged for administration in a single dose.

By “subunit” is meant to include a composition, mixture, particle, etc., that can provide an oral dosage form alone or when combined with other subunits. By “part of the same subunit” is meant to refer to a subunit comprising certain ingredients.

By “releasable form” is meant to include immediate-release, controlled-release, delayed-release, pulsed-release, and sustained-release forms. Certain release forms can be characterized by their dissolution profile. “Dissolution profile” as used herein, means a plot of the cumulative amount of active ingredient released as a function of time. The dissolution profile can be measured utilizing the Drug Release Test <724>, which incorporates standard test USP 26 (Test <711> Dissolution). A profile is characterized by the test conditions selected. Thus the dissolution profile can be generated at a preselected apparatus type, shaft speed, temperature, volume, and pH of the dissolution media.

Several dissolution profiles can be obtained. For example, a first dissolution profile can be measured at a pH level approximating that of the stomach; a second dissolution profile can be measured at a pH level approximating that of one point in the intestine or several pH levels approximating multiple points in the intestine. Another dissolution profile can be measured using purified water.

A highly acidic pH may simulate the stomach and a less acidic to basic pH can simulate the intestine. By the term “highly acidic pH” it is meant a pH of about 1 to about 4. By the term “less acidic to basic pH” is meant a pH of greater than about 4 to about 7.5, preferably about 6 to about 7.5. A pH of about 1.2 can be used to simulate the pH of the stomach. A pH of about 6.0 to about 7.5, preferably about 6.8 can be used to simulate the pH of the intestine.

Release forms may also be characterized by their pharmacokinetic parameters. “Pharmacokinetic parameters” are parameters which describe the in vivo characteristics of the active agent over time, including for example plasma concentration of the active agent. By “Cₘₐₓ” is meant the measured concentration of the active agent in the plasma at the point of maximum concentration. By “C₂₄” is meant the concentration of the active agent in the plasma at about 24 hours. The term “Tₘₐₓ” refers to the time at which the concentration of the active agent in the plasma is the highest. “AUC” is the area under the curve of a graph of the concentration of the active agent (typically plasma concentration) vs. time, measured from one time to another.

By “sequestered form” is meant an ingredient that is not released or substantially not released at one hour after the intact dosage form comprising the active agent is orally administered. The term “substantially not released” is meant to include the ingredient that might be released in a small amount, as long as the amount released does not affect or does not significantly affect efficacy when the dosage form is orally administered to mammals, for example, humans, as intended.

By “instant-release” is meant a dosage form designed to ensure rapid dissolution of the active agent by modifying the normal crystal form of the active agent to obtain a more rapid dissolution.

By “immediate-release”, it is meant a conventional or non-modified release form in which greater then or equal to about 75% of the active agent is released within two hours of administration, preferably within one hour of administration.

By “controlled-release” it is meant a dosage form in which the release of the active agent is controlled or modified over a period of time. Controlled can mean, for example, sustained, delayed or pulsed-release at a particular time. Alternatively, controlled can mean that the release of the active agent is extended for longer than it would be in an immediate-release dosage form, i.e., at least over several hours.

By “sustained-release” or “extended-release” is meant to include the release of the active agent at such a rate that blood (e.g., plasma) levels are maintained within a therapeutic range but below toxic levels for at least about 8 hours, preferably at least about 12 hours after administration at steady-state. The term “steady-state” means that a plasma level for a given active agent has been achieved and which is maintained with subsequent doses of the drug at a level which is at or above the minimum effective therapeutic level and is below the minimum toxic plasma level for a given active agent. With regard to dissolution profiles, the first and second dissolution profiles (e.g., in the stomach and in the intestines) should each be equal to or greater than the minimum dissolution required to provide substantially equivalent bioavailability to a capsule, tablet or liquid containing the at least one active ingredient in an immediate-release form.

By “delayed-release”, it is meant that there is a time-delay before significant plasma levels of the active agent are achieved. A delayed-release formulation of the active agent can avoid an initial burst of the active agent, or can be formulated so that release of the active agent in the stomach is avoided and absorption is effected in the small intestine.

A “pulsed-release” formulation can contain a combination of immediate-release, sustained-release, and/or delayed-release formulations in the same dosage form. A “semi-delayed-release” formulation is a pulsed-released formulation in which a moderate dosage is provided immediately after administration and a further dosage some hours after administration.

Certain formulations described herein may be “coated”. The coating can be a suitable coating, such as, a functional or a non-functional coating, or multiple functional and/or non-functional coatings. By “functional coating” is meant to include a coating that modifies the release properties of the total formulation, for example, a sustained-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

By “REMINYL” is meant galantamine hydrobromide formulations manufactured by JOLLC, Gurabo, Puerto Rico or Janssen-Cilag SpA Latina, Italy (tablets); or Janssen Pharmaceutica N.V. Beerse, Belgium (oral solution). Preferably, by REMINYL is meant film coated tablets of galantamine hydrobromide, base equivalent of 4, 8, and 12 mg in the presence of inactive ingredients of colloidal silicon dioxide, crospovidone, hydroxy propyl methylcellulose, lactose monohydrate, magnesium stearate, microcrystalline cellulose, propylene glycol, talc, and titanium dioxide, optionally yellow ferric oxide (4 mg tablet), red ferric oxide (8 mg tablet), or red ferric oxide and FD&C yellow #6 aluminum lake (12 mg tablet).

In some embodiments, the formulations described herein preferably exhibit bioequivalence to the marketed drug product, for example REMINYL. Bioequivalence is defined as “the absence of a significant difference in the rate and extent to which the active ingredient or active moiety in pharmaceutical equivalents or pharmaceutical alternatives becomes available at the site of drug action when administered at the same molar dose under similar conditions in an appropriately designed study” (21 CFR 320.1). As used herein, bioequivalence of a dosage form is determined according to the Federal Drug Administration's (FDA) guidelines and criteria, including “GUIDANCE FOR INDUSTRY BIOAVAILABILITY AND BIOEQUVALENCE STUDIES FOR ORALLY ADMINISTERED DRUG PRODUCTS—GENERAL CONSIDERATIONS” available from the U.S. Department of Health and Human Services (DHHS), Food and Drug Administration (FDA), Center for Drug Evaluation and Research (CDER) March 2003 Revision 1; and “GUIDANCE FOR INDUSTRY STATISTICAL APPROACHES TO ESTABLISHING BIOEQUIVALENCE” DHHS, FDA, CDER, January 2001; and “STATISTICAL PROCEDURES FOR BIOEQUIVALENCE STUDIES USING A STANDARD TWO-TREATMENT CROSSOVER DESIGN” DHHS, FDA, CDER, July 1992, all of which are incorporated herein in their entirety. Particularly relevant sections of the guidelines include:

Pharmacokinetic Analysis of Data: Calculation of area under the plasma concentration-time curve to the last quantifiable concentration (AUC₀₋ₜ,) and to infinity (AUCO_{0-∞}), Cₘₐₓ, and Tₘₐₓ should be performed according to standard techniques.

Statistical Analysis of Pharmacokinetic Data: The log transformed AUC and Cₘₐₓ data should be analyzed statistically using analysis of variance. These two parameters for the test product should be shown to be within 80-125% of the reference product using the 90% confidence interval. See also Division of Bioequivalence Guidance Statistical Procedures for Bioequivalence Studies Using a Standard Two-Treatment Crossover Design.

Multiple Dose Studies: At a minimum, the following pharmacokinetic parameters for the substance of interest should be measured in a multiple dose bioequivalence study:
a. Area under the plasma/blood concentration—time curve from time zero to time T over a dosing interval at steady state (AUC_{0-T}), wherein T is the dosing interval.
b. Peak drug concentration (Cₘₐₓ) and the time to peak drug concentration (Tₘₐₓ), obtained directly from the data without interpolation, after the last dose is administered.
c. Drug concentrations at the end of each dosing interval during steady state (Cₘᵢₙ).
d. Average drug concentration at steady state (Cₐᵥ), where Cₐᵥ−AUC_{0-T}/T.
e. Degree of fluctuation (DF) at steady state, where DF=100% X (Cₘₐₓ−Cₘᵢₙ)/Cₐᵥ. Evidence of attainment of steady state for the test and reference products should be submitted in the bioequivalence study report.

Statistical Analysis Parametric (normal-theory) general linear model procedures are recommended for the analysis of pharmacokinetic data derived from in vivo bioequivalence studies. An analysis of variance (ANOVA) should be performed on the pharmacokinetic parameters AUC and Cmax using General Linear Models (GLM) procedures of SAS (4) or an equivalent program. Appropriate statistical models pertaining to the design of the bioequivalence study should be employed. For example, for a conventional two-treatment, two-period, two-sequence (2×2) randomized crossover study design, the statistical model often includes factors accounting for the following sources of variation:
1. Sequence (sometimes called Group or Order)
2. Subjects, nested in sequences
3. Period (or Phase)
4. Treatment (sometimes called Drug or Formulation) =p The sequence effect should be tested using the [subject (sequence)]mean square from the ANOVA as an error term. All other main effects should be tested against the residual error (error mean square) from the ANOVA. The LSMEANS statement should be used to calculate least squares means for treatments. The ESTIMATE statement in SAS should be used to obtain estimates for the adjusted differences between treatment means and the standard error associated with these differences.

The two one-sided hypotheses at the α=0.05 level of significance should be tested for AUC and Cₘₐₓ by constructing the 90% confidence interval for the ratio between the test and reference averages.

Logarithmic Transformation of Pharmacokinetic Data:

Statistical Assumptions: The assumptions underlying the ANOVA are:
1. Randomization of samples
2. Homogeneity of variances
3. Additivity (linearity) of the statistical model
4. Independency and normality of residuals

In bioequivalence studies, these assumptions can be interpreted as follows:
1. The subjects chosen for the study should be randomly assigned to the sequences of the study.
2. The variances associated with the two treatments, as well as between the sequence groups, should be equal or at least comparable.
3. The main effects of the statistical model, such as 25 subject, sequence, period and treatment effect for a standard 2×2 crossover study, should be additive. There should be no interactions between these effects.
4. The residuals of the model should be independently and normally distributed. In other words, data from bioequivalence studies should have a normal distribution.

If these assumptions are not met, additional steps should be taken prior to the ANOVA including data transformation to improve the fit of the assumptions or use of a nonparametric statistical test in place of ANOVA. However, the normality and constant variance assumptions in the ANOVA model are known to be relatively robust, i.e., small or moderate departure from each (or both) of these assumptions will not have a significant effect on the final result.

The formulations that exhibit bioequivalence to REMINYL are those wherein the formulations provide an AUC after administration that is more than 80 percent and less than 120 percent of the AUC provided between 0 and 24 hours (optionally between 0 and 36 hours) after administration by the same strength dosage form of galantamine hydrobromide wherein the same strength dosage form of galantamine hydrobromide comprises colloidal silicon dioxide in a weight ratio to galantamine hydrobromide of about 0.0234:1, crospovidone in a weight ratio to galantamine hydrobromide of about 0.585:1, hydroxypropyl methylcellulose in a weight ratio to galantamine hydrobromide of about 0.488:1, lactose monohydrate in a weight ratio to galantamine hydrobromide of about 7.53:1, magnesium stearate in a weight ratio to galantamine hydrobromide of about 0.0585:1, microcrystalline cellulose in a weight ratio to galantamine hydrobromide of about 2.51:1, propylene glycol in a weight ratio to galantamine hydrobromide of about 0.188:1, talc in a weight ratio to galantamine hydrobromide of about 0.0975:1, and titanium dioxide in a weight ratio to galantamine hydrobromide of about 0.146:1.

Disclosed herein is a dosage formulation comprises galantamine or a pharmaceutically acceptable salt thereof; and a pharmaceutically acceptable carrier, wherein the carrier is substantially free of a spray dried mixture of lactose monohydrate and microcrystalline cellulose; and wherein the formulation exhibits a dissolution profile such that after 0.5 hour at least about 80% of the galantamine or galantamine salt is released after combining the dosage form with 500 ml of purified water at 37° C. in Apparatus 2 (USP, <711 > Dissolution, paddle, 50 rpm). Preferably the formulation is in the form of a tablet. Within this embodiment, it may be preferred to have a formulation that exhibits a dissolution profile such that after 0.5 hour at least about 82%, about 84%, about 86%, about 88%, about 90%, or even about 95% of the galantamine or galantamine salt is released. The procedure employed to obtain the dissolution profile may be obtained from USP 23, for example.

Carriers for the formulation are inert substances that may be used as a vehicle for the active agent, optionally in conjunction with other excipients, as long as the resulting formulation meets the dissolution profile desired. Suitable carriers are known in the art and include, for example, microcrystalline cellulose, anhydrous lactose, starches, powdered cellulose, dextrates, dextrin, dextrose, fructose, kaolin, lactitol, mannitol, sorbitol, and the like.

A “disintegrant” is meant an agent used in a formulation, (e.g. tablet or capsule) to aid in the break down of a compacted mass in the presence of a fluid environment. Compounds that behave as disintegrants generally possess the ability to swell or expand upon exposure to the fluid environment. Preferably the disintegrant swells upon exposure to an aqueous environment. Certain traditional tablet fillers may function as a disintegrant (e.g. starch and microcrystalline cellulose), other materials provide superior results as a disintegrant, for example, croscarmellose sodium, crospovidone, low-substituted hydroxypropyl cellulose, sodium starch glycolate, and the like. Other suitable disintegrants include sodium carboxymethyl cellulose, alginates, and the like.

Dosage Forms: Release Properties

The dosage forms comprising the active agent can be characterized by the release properties of the formulation. Certain dosage form can be targeted-release formulations wherein release occurs in a particular segment of the gastrointestinal tract, for example in the small intestine.

Targeted-Release Dosage Forms

Targeted-release refers to release of galantamine in a particular segment of the gastrointestinal tract. A targeted-release formulation may, for example, have a coat such as an enteric coat wherein release to a particular portion of the gastrointestinal tract is achieved by the coat. In addition to coatings, other ingredients or techniques may be used to enhance the absorption of the active agent, to improve the disintegration profile, and/or to improve the properties of the active agent and the like. These include, but are not limited to, the use of additional chemical penetration enhancers, which are referred to herein as noneffervescent penetration enhancers; absorption of the active agent onto fine particles to promote absorption by specialized cells within the gastrointestinal tract (such as the M cells of Peyer's patches); ion pairing or complexation; and the use of lipid and/or surfactant active agent carriers. The selected enhancement technique is related to the route of active agent absorption, i.e., paracellular or transcellular.

A bioadhesive polymer may be included in the oral dosage form to increase the contact time between the dosage form and the mucosa of the most efficiently absorbing section of the gastrointestinal tract. Nonlimiting examples of known bioadhesives include carbopol (various grades), sodium carboxy methylcellulose, methylcellulose, polycarbophil (NOVEON AA-1), hydroxypropyl methylcellulose, hydroxypropyl cellulose, sodium alginate, sodium hyaluronate, and combinations comprising one or more of the foregoing bioadhesives.

Disintegration agents may also be employed to aid in dispersion of galantamine in the gastrointestinal tract. Disintegration agents may be pharmaceutically acceptable effervescent agents. In addition to the effervescence-producing disintegration agents, a dosage form may include suitable noneffervescent disintegration agents. Nonlimiting examples of disintegration agents include microcrystalline cellulose, croscarmellose sodium, crospovidone, sodium starch glycollate, starches and modified starches, and combinations comprising one or more of the foregoing disintegration agents.

Apart from any effervescent material within the tablet, additional effervescent components or, alternatively, only sodium bicarbonate (or other alkaline substance) may be present in the coating around the dosage form. The purpose of the latter effervescent/alkaline material is to react within the stomach contents and promote faster stomach emptying.

Enteric-Coated Formulations

An enteric coating is a coating that prevents release of the active agent until the dosage form reaches the small intestine. Enteric-coated dosage forms comprise active agent coated with an enteric polymer. The enteric polymer should be non-toxic and is predominantly soluble in the intestinal fluid, but substantially insoluble in the gastric juices. Examples include polyvinyl acetate phthalate (PVAP), hydroxypropylmethyl-cellulose acetate succinate (HPMCAS), cellulose acetate phthalate (CAP), methacrylic acid copolymer, hydroxy propyl methylcellulose succinate, cellulose acetate succinate, cellulose acetate hexahydrophthalate, hydroxypropyl methylcellulose hex ahydrophthal ate, hydroxypropyl methylcellulose phthalate (HPMCP), cellulose propionate phthalate, cellulose acetate maleate, cellulose acetate trimellitate, cellulose acetate butyrate, cellulose acetate propionate, methacrylic acid/methacrylate polymer, methacrylic acid-methyl methacrylate copolymer, ethyl methacrylate-methylmethacrylate-chlorotrimethylammonium ethyl methacrylate copolymer, and the like, and combinations comprising one or more of the foregoing enteric polymers. Other examples include natural resins, such as shellac, SANDARAC, copal collophorium, and combinations comprising one or more of the foregoing polymers. Yet other examples of enteric polymers include synthetic resin bearing carboxyl groups. The methacrylic acid: acrylic acid ethyl ester 1:1 copolymer solid substance of the acrylic dispersion sold under the trade designation “EUDRAGIT L-100-55” has been found to be suitable.

Immediate-Release Dosage Forms

An immediate-release dosage form is one in which the release properties of the drug from the dosage form are essentially unmodified. An immediate-release dosage form preferably results in delivery of greater then or equal to about 75% galantamine within about 2 hours of administration, preferably within 1 hour of administration. An immediate-release dosage form may contain optional excipients so long as the excipients do not significantly extend the release time of the drug.

SUSTAINED-RELEASE DOSAGE FORMS

A sustained-release form is a form suitable for providing controlled-release of galantamine over a sustained period of time (e.g., 8 hours, 12 hours, 24 hours). Sustained-release dosage forms may release the active agent at a rate independent of pH, for example, about pH 1.2 to about 7.5. Alternatively, sustained-release dosage forms may release the active agent at a rate dependent upon pH, for example, a lower rate of release at pH 1.2 and a higher rate of release at pH 7.5. Preferably, the sustained-release form avoids “dose dumping” upon oral administration. The sustained-release oral dosage form can be formulated to provide for an increased duration of therapeutic action allowing once-daily dosing.

A sustained-release dosage form comprises a release-retarding material. The release-retarding material can be, for example, in the form of a matrix or a coating. The active agent in sustained-release form may be, for example, a particle of the active agent that is combined with a release-retarding material. The release-retarding material is a material that permits release of the active agent at a sustained rate in an aqueous medium. The release-retarding material can be selectively chosen so as to achieve, in combination with the other stated properties, a desired in vitro release rate.

Release-retarding materials can be hydrophilic and/or hydrophobic polymers. Release-retarding materials include, for example acrylic polymers, wax, alkylcelluloses, shellac, zein, hydrogenated vegetable oil, hydrogenated castor oil, and combinations comprising one or more of the foregoing materials. The oral dosage form can contain between about 1% and about 80% (by weight) of the release-retarding material. Suitable acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, and combinations comprising one or more of the foregoing polymers. The acrylic polymer may comprise a methacrylate copolymers described in NF XXIV as fully polymerized copolymers of acrylic and methacrylic acid esters with a low content of quaternary ammonium groups.

Suitable modified celluloses include, for example, alkyl celluloses and hydroxyalkyl celluloses. Alkyl cellulose includes, for example, methyl cellulose, ethyl cellulose, and the like. Hydroxyalkyl cellulose includes, for example, hydroxyethyl cellulose, hydroxypropyl cellulose, hydroxypropylmethyl cellulose, hydroxypropylethyl cellulose, hydroxypropylpropyl cellulose, hydroxypropylbutyl cellulose, and the like. Those skilled in the art will appreciate that other cellulosic polymers, including other alkyl or hydroxyalkyl cellulosic polymers, can be substituted for part or all of the ethyl cellulose and/or hydroxyalkyl cellulose.

Other suitable hydrophobic materials are water-insoluble with more or less pronounced hydrophobic trends. The hydrophobic material may have a melting point of about 30° C. to about 200° C., more preferably about 45° C. to about 90° C. The hydrophobic material can include neutral or synthetic waxes, fatty alcohols (such as lauryl, myristyl, stearyl, cetyl or preferably cetostearyl alcohol), fatty acids, including fatty acid esters, fatty acid glycerides (mono-, di-, and tri-glycerides), hydrogenated fats, hydrocarbons, normal waxes, stearic acid, stearyl alcohol, hydrophobic and hydrophilic materials having hydrocarbon backbones, and combinations comprising one or more of the foregoing materials. Suitable waxes include beeswax, glycowax, castor wax, carnauba wax and wax-like substances, e.g., material normally solid at room temperature and having a melting point of from about 30° C. to about 100° C., and combinations comprising one or more of the foregoing waxes.

In other embodiments, the release-retarding material may comprise digestible, long chain (e.g., C₈-C₅₀, preferably C₁₂-C₄₀), substituted or unsubstituted hydrocarbons, such as fatty acids, fatty alcohols, glyceryl esters of fatty acids, mineral and vegetable oils, waxes, and combinations comprising one or more of the foregoing materials. Hydrocarbons having a melting point of between about 25° C. and about 90° C. may be used. Of these long chain hydrocarbon materials, fatty (aliphatic) alcohols are preferred. The oral dosage form can contain up to about 60% by weight of at least one digestible, long chain hydrocarbon.

Further, the sustained-release matrix can contain up to 60% by weight of at least one polyalkylene glycol.

Alternatively, the release-retarding material may comprise polylactic acid, polyglycolic acid or a co-polymer of lactic and glycolic acid.

Release-modifying agents, which affect the release properties of the release-retarding material, may optionally be used. The release-modifying agent may, for example, function as a pore-former. The pore former can be organic or inorganic, and include materials that can be dissolved, extracted or leached from the coating in the environment of use. The pore-former can comprise one or more hydrophilic polymers, such as hydroxypropylmethylcellulose, hydroxypropylcellulose, polycarbonates comprised of linear polyesters of carbonic acid in which carbonate groups reoccur in the polymer chain, and combinations comprising one or more of the foregoing release-modifying agents. Alternatively, the pore former may be a small molecule such as lactose, or metal stearates, and combinations comprising one or more of the foregoing release-modifying agents.

The release-retarding material can also optionally include other additives such as an erosion-promoting agent (e.g., starch and gums); and/or a semi-permeable polymer. In addition to the above ingredients, a sustained-release dosage form may also contain suitable quantities of other materials, e.g., diluents, lubricants, binders, granulating aids, colorants, flavorants and glidants that are conventional in the pharmaceutical art. The release-retarding material can also include an exit means comprising at least one passageway, orifice, or the like. The passageway can have any shape, such as round, triangular, square, elliptical, irregular, etc.

The sustained-release dosage form comprising an active agent and a release-retarding material may be prepared by a suitable technique for preparing active agents as described in detail below. The active agent and release-retarding material may, for example, be prepared by wet granulation techniques, melt extrusion techniques, etc. To obtain a sustained-release dosage form, it may be advantageous to incorporate an additional hydrophobic material.

The active agent in sustained-release form can include a plurality of substrates comprising the active ingredient, which substrates are coated with a sustained-release coating comprising a release-retarding material. The sustained-release preparations may thus be made in conjunction with a multiparticulate system, such as beads, ion-exchange resin beads, spheroids, microspheres, seeds, pellets, granules, and other multiparticulate systems in order to obtain a desired sustained-release of the active agent. The multiparticulate system can be presented in a capsule or other suitable unit dosage form.

In certain cases, more than one multiparticulate system can be used, each exhibiting different characteristics, such as pH dependence of release, time for release in various media (e.g., acid, base, simulated intestinal fluid), release in vivo, size, and composition.

In some cases, a spheronizing agent, together with the active ingredient can be spheronized to form spheroids. Microcrystalline cellulose and hydrous lactose impalpable are examples of such agents. Additionally (or alternatively), the spheroids can contain a water insoluble polymer, preferably an acrylic polymer, an acrylic copolymer, such as a methacrylic acid-ethyl acrylate copolymer, or ethyl cellulose. In this formulation, the sustained-release coating will generally include a water insoluble material such as a wax, either alone or in admixture with a fatty alcohol, or shellac or zein.

Spheroids or beads, coated with an active ingredient can be prepared, for example, by dissolving or dispersing the active ingredient in a solvent and then spraying the solution onto a substrate, for example, sugar spheres NF-21, 18/20 mesh, using a Wurster insert. Optionally, additional ingredients are also added prior to coating the beads in order to assist the active ingredient binding to the substrates, and/or to color the resulting beads, etc. The resulting substrate-active material may optionally be overcoated with a barrier material, to separate the therapeutically active agent from the next coat of material, e.g., release-retarding material. Preferably, the barrier material is a material comprising hydroxypropylmethylcellulose. However, any film-former known in the art may be used. Preferably, the barrier material does not affect the dissolution rate of the final product

To obtain a sustained-release of the active agent in a manner sufficient to provide an effect for the sustained durations, the substrate comprising the active agent can be coated with an amount of release-retarding material sufficient to obtain a weight gain level from about 2 to about 30%, although the coat can be greater or lesser depending upon the physical properties of the active agent utilized and the desired release rate, among other things. Moreover, there can be more than one release-retarding material used in the coat, as well as various other pharmaceutical excipients.

The release-retarding material may thus be in the form of a film coating comprising a dispersion of a hydrophobic polymer. Solvents typically used for application of the release-retarding coating include pharmaceutically acceptable solvents, such as water, methanol, ethanol, methylene chloride, and combinations comprising one or more of the foregoing solvents.

In addition, the sustained-release profile of active agent release in the formulations (either in vivo or in vitro) can be altered, for example, by using more than one release-retarding material, varying the thickness of the release-retarding material, changing the particular release-retarding material used, altering the relative amounts of release-retarding material, altering the manner in which the plasticizer is added (e.g., when the sustained-release coating is derived from an aqueous dispersion of hydrophobic polymer), by varying the amount of plasticizer relative to retardant material, by the inclusion of additional ingredients or excipients, by altering the method of manufacture, etc.

In addition to or instead of being present in a matrix, the release-retarding agent can be in the form of a coating. Optionally, the dosage forms can be coated, or a gelatin capsule can be further coated, with a sustained-release coating such as the sustained-release coatings described herein. Such coatings are particularly useful when the subunit comprises the active agent in releasable form, but not in sustained-release form. The coatings preferably include a sufficient amount of a hydrophobic material to obtain a weight gain level from about 2 to about 30 percent, although the overcoat can be greater upon the physical properties of the particular the active agent and the desired release rate, among other things.

The sustained-release formulations preferably slowly release the active agent, e.g., when ingested and exposed to gastric fluids, and then to intestinal fluids. The sustained-release profile of the formulations can be altered, for example, by varying the amount of retardant, e.g., hydrophobic material, by varying the amount of plasticizer relative to hydrophobic material, by the inclusion of additional ingredients or excipients, by altering the method of manufacture, etc.

In a preferred embodiment, the sustained-release formulation comprises the active agent and a release-retarding material such that the formulation exhibits a dissolution profile such that less than about 18% of the galantamine or galantamine hydrobromide is released in 1 hour, and less than about 80% of the galantamine or galantamine hydrobromide is released in 10 hours in USP buffer pH 6.8 at 37° C. in an Apparatus 2 (USP, <711> Dissolution, paddle, 50 rpm).

In another embodiment, the sustained-release formulation comprises a first subunit wherein the first subunit comprises a galantamine or a pharmaceutically acceptable salt thereof, and a first release-retarding material; and a second subunit, wherein the second subunit comprises galantamine or a pharmaceutically acceptable salt thereof, and a second release-retarding material, wherein the first and second release-retarding material can be the same or different, and wherein the dosage formulation, at steady-state, provides a maximum galantamine plasma concentration (Cₘₐₓ) and an galantamine plasma concentration at about 24 hours after administration (C₂₄), wherein the ratio of Cₘₐₓ to C₂₄ is less than about 4:1.

Delayed-Release Dosage Forms

Delayed-release tablets may comprise a core, a first coating and optionally a second coating. The core may include the active agent, and excipients, notably a lubricant, and a binder and/or a filler, and optionally a glidant as well as other excipients.

Examples of suitable lubricants include stearic acid, magnesium stearate, glyceryl behenate, talc, mineral oil (in PEG), etc. Examples of suitable binders include water-soluble polymer, such as modified starch, gelatin, polyvinylpyrrolidone, polyvinyl alcohol, etc. Examples of suitable fillers include lactose, microcrystalline cellulose, etc. An example of a glidant is silicon dioxide (AEROSIL, Degussa).

The core may contain, by dry weight, about 1 to about 25% active agent or a pharmaceutically acceptable salt thereof, about 0.5 to about 10% lubricant, and about 25 to about 98% binder or filler.

The first coating may be, for example, a semi-permeable coating to achieve delayed-release of the active agent. The first coating may comprise a water-insoluble, film-forming polymer, together with a plasticizer and a water-soluble polymer. The water-insoluble, film-forming polymer can be a cellulose ether, such as ethylcellulose, a cellulose ester, such as cellulose acetate, polyvinylalcohol, etc. A suitable film-forming polymer is ethylcellulose (available from Dow Chemical under the trade name ETHOCEL). Other excipients can optionally also be present in the first coating, as for example acrylic acid derivatives (such and EUDRAGIT, Rohm Pharma), pigments, etc.

The first coating contains from about 20 to about 85% water-insoluble, polymer (e.g. ethylcellulose), about 10 to about 75% water-soluble polymer (e.g. polyvinylpyrrolidone), and about 5 to about 30% plasticizer. The relative proportions of ingredients, notably the ratio of water-insoluble, film-forming polymer to water-soluble polymer, can be varied depending on the release profile to be obtained (where a more delayed-release is generally obtained with a higher amount of water-insoluble, film-forming polymer).

The weight ratio of first coating to tablet core can be about 1:30 to about 3:10, preferably about 1:10.

The optional second coating may be designed to protect the coated tablet core from coming into contact with gastric juice, thereby preventing a food effect. The second coating may comprises an enteric polymer of the methacrylic type and optionally a plasticizer. The second coating can contain, by weight, about 40 to about 95% enteric polymer (e.g. EUDRAGIT L30D-55) and about 5 to about 60% plasticizer (e.g. triethyl citrate, polyethylene glycol). The relative proportions of ingredients, notably the ratio methacrylic polymer to plasticizer can be varied according to a methods known to those of skill in the art of pharmaceutical formulation.

A process for preparing a delayed-release dosage form of the active agent comprises manufacturing a core by, for example, wet or dry granulation techniques. Alternatively, the active agent and lubricant may be mixed in a granulator and heated to the melting point of the lubricant to form granules. This mixture can then be mixed with a suitable filler and compressed into tablets. Alternatively, the active agent and a lubricant (e.g. mineral oil in PEG) may be mixed in a granulator, e.g. a fluidized bed granulator and then into tablets. Tablets may be formed by standard techniques, e.g. on a (rotary) press (for example KILIAN) fitted with suitable punches. The resulting tablets are hereinafter referred as tablet cores.

The coating process can be as follows. Ethylcellulose and polyethylene glycol (e.g. PEG 1450) are dissolved in a solvent such as ethanol; polyvinylpyrrolidone is then added. The resulting solution is sprayed onto the tablet cores, using a coating pan or a fluidized bed apparatus.

The process for applying the second coating can be as follows. Triethyl citrate and polyethylene glycol (e.g. PEG 1450) are dissolved in a solvent such as water; methacrylic polymer dispersion is then added. If present, silicon dioxide can be added as a suspension. The resulting solution is sprayed onto the coated tablet cores, using a coating pan or a fluidized bed apparatus.

The weight ratio of the second coating to coated tablet core is about 1:30 to about 3:10, preferably about 1:10.

An exemplary delayed-release dosage form comprises a core containing the active agent, polyvinylalcohol and glyceryl behenate; a first coating of ethylcellulose, polyvinylpyrrolidone and polyethylene glycol, and a second coating of methacrylic acid co-polymer type C, triethyl citrate, polyethylene glycol and optionally containing silicon dioxide.

Additional Delayed or Extended Release Forms

A delayed-release formulation may employ an ion exchange resin chosen specifically for the type of active agent in the formulation. Specifically, the formulation can be a multiparticulate composition wherein each particle includes the active agent reversibly adsorbed onto an ion exchange material to form an active agent-resin complex, and each particle is coated with a polymeric coating material. The ion exchange resin can be either a cation exchange resin or an anion exchange resin. The formulation is based on loading the active agent onto an ion exchange resin of opposite charge, coating the discrete resin particles with a coating, and incorporating the resulting coated active agent loaded resin particles into a convenient oral dosage form.

The oral dosage forms can be either solid or liquid oral dosage forms. Convenient oral dosage forms such as suspensions, syrups, sprinkles, fast melt tablets, effervescent tablets and fast dissolving tablets are readily acceptable to patients resulting in increased patient compliance for a given therapeutic regimen.

Ion-exchange resin materials include any ion-exchange resin which is capable of binding the active agent, including, for instance, anionic and cationic resin materials. Where the active agent is a cation or is prone to protonation, the ion-exchange resin is suitably a cation exchange resin material. That is to say, a resin having a predominantly negative charge along the resin backbone, or a resin having a pendant group suitable for cation exchange, and which has an affinity for positively charged ions or cationic species. Typical of such cation exchange resins include resins having polymer backbones comprising styrene-divinyl benzene colpolymers, methacrylic acid and divinyl benzene co-polymers, and resins with pendant functional groups suitable for cation exchange, such as sulfonate and carboxylate groups. Cation exchange resins suitable for use in the practice of the present invention include for example those sold under the trade names Amberlite IRP-64, Amberlite IRP-69 and Amberlite IRP 88 (Rohm and Haas, Frankfurt, Germany), Dowex 50V,TX2-400, Dowex 50WX4-400 and Dowex 50WX8400 (The Dow Chemical Company, Midland, Mich.), Purolite C 115HMR and Purolite C 102DR (Purolite International Ltd., Hounslow, Great Britain).

Similarly, where the drug is an anion or is prone to deprotonation, the ion exchange resin is suitably an anion exchange resin material. That is to say, a resin having a predominantly positive charge along the resin backbone, or a resin having a pendant group suitable for anion exchange, and which has an affinity for negatively charged ions or anionic species. Typical of such anion exchange resins include resins having polymer backbones comprising styrene, acrylic acid or phenol units, co-polymers thereof, styrene-divinyl benzene co-polymers and phenolic-based polyamine condensates and resins with pendant functional groups suitable for anion exchange, such as ammonium or tetraalkyl ammonium functional groups. Anion exchange resins suitable for use in the practice of the present invention include for example those sold under the trade names Amberlite IRP-58, Amberlite RA-67, Amberlite IRA 68 (Rohm and Haas, Frankfurt, Germany), Dowex 1X2-400, Dowex 1X4-400, Dowex 1X8400 and Dowex 2X8400 (The Dow Chemical Company, Midland, Mich), Purolite A845, Purolite A500P and Purolite PCA-433 (Purolite International Ltd., Hounslow, Great Britain), Duolite AP 143/1092 and Duolite A 143/1093.

Ion exchange resins with various degrees of crosslinking and a range of binding capacities may also be used.

The oral dosage forms of the present invention can be prepared by contacting the ion exchange resin with the active ingredient to form an active ingredient or drug/ion exchange resin core or complex. The individual cores can then be coated with the polymeric coating material.

Typically the ion exchange resins suitable for use in the present invention are in the form of ion exchange resin particles. Stirring the ion-exchange resin particles in a solution of the selected active agent is usually sufficient to achieve binding of the active agent onto the resin particles.

Loading of the resin is suitably carried out at a pH that facilitates binding of the active agent compound. Some ion exchange resins may require “activation” by rinsing with a solution of acid or base, prior to loading with the drug. Such activation requirements will be well known to those skilled in the art of working with ion exchange resin materials. Specific requirements for individual ion exchange resin materials may be obtained from the resin manufactures. Preferably, the particles are spherical to enable substantially complete coating of the particle.

The term “reversibly adsorb” means that the drug binds to an ion exchange resin of opposite charge via an ionic interaction that can be reversed in suitable ionic conditions. The active ingredient component of the composition may be present in any amount which is sufficient to elicit a therapeutic effect. Typically the active ingredient is present at about 1 to about 70% by weight of the uncoated resin, preferably the active agent ranges from about 5 to about 60% by weight of the uncoated resin, more preferably the active ingredient ranges from about 10 to about 50%, and yet more preferably about 10 to about 40%, by weight of the uncoated resin.

The polymer material used for coating the drug-resin complex can be a polymer that has properties which can prevent the release of the drug until it reaches a specific site in the gastrointestinal tract and only then the drug is released. The specific site in the gastrointestinal tract includes any point in the tract including the esophagus, the stomach, and the intestine.

The polymeric coating can be a pH dependent or independent polymer and can include a combination or two or more polymeric materials. Suitable pH independent coating materials include, for example, alkyl celluloses such as methyl cellulose, hydroxyalkyl alkyl celluloses such as hydroxy propyl methyl cellulose, hydroxy alkyl celluloses such as hydroxy propyl cellulose and hydroxy ethyl cellulose, polyvinyl alcohol, maltodextrin, polymethacrylates such as Eudragit RL (Rohm-Pharma, Darmstadt, Germany). Suitable pH dependent coating materials include, for example, esters of at least one cellulose derivative such as an alkyl cellulose, a hydroxyalkyl cellulose, a hydroxyalkyl alkyl cellulose or a cellulose ester, with at least one polybasic acid such as succinic acid, maleic acid, phthalic acid, tetrahydrophthalic acid, hexahydrophthalic acid, trimellitic acid or pyromellitic acid. Suitable enteric coating materials include for example those selected from the group consisting of hydroxy propyl methyl cellulose phthalate (HPMCP), cellulose acetate phthalate (CAP), cellulose acetate trimillitate (CAT) and hydroxypropyl methylcellulose acetate succinate. Also considered useful in the practice of the present invention are for example enteric materials such as those selected from the group consisting of poly vinyl acetate phthalate (PVAP), polyvinyl acetaldiethylamino acetate, and shellac. Particularly useful are poly acrylic and methacrylic acids and poly acrylate and methacrylate based coatings, and mixtures thereof, such as those sold under the tradename Eudragit, for example Eudragit L, and Eudragit S (Rohm-Pharma, GmbH, Darmstadt, Germany) to 50-250% by weight of the drug loaded resin particles. A further particularly useful pH dependent coating material suitable for use in accordance with the present invention is Eudragit E (Rohm-Pharma, Darmstadt, Germany).

A particularly useful pH independent polymer for use in accordance with the present invention is Eudragit RD 100 (Rohm-Pharma, Darmstadt, Germany). The polymeric coating may suitably comprise a combination of two or more polymer materials.

The coating may be applied to the drug loaded particles by any suitable technique. Such techniques will be apparent to those skilled in the art. Particularly useful for application of the coating is the technique of spray coating, carried out for instance using a fluidized bed coating apparatus. Suitable excipients and/or additives may be added to the coating formulations. For example it may be desirable to add plasticisers, glidants, anti-tacking agents, pigments and other excipients to the coating formulations. Suitable plasticisers include, for example, triethyl citrate and polyethylene glycol. Suitable glidants include, for example, talc, glycerol monostearate and magnesium stearate.

The coating material may be applied to the drug loaded particles in any amount. Typically the coating material is applied in an amount equivalent to about 10 to about 300% by weight of the drug-loaded resin particles. Preferably, the coating material is applied in an amount equivalent to about 20 to about 250% by weight of the drug loaded resin particles.

Drug loaded, coated resin particles of any size suitable for incorporation into a final dosage form may be used. Typically drug loaded, coated resin particles making up the multiparticulate composition of the present invention have an average diameter (defined as D50%) of 20-750 micrometer.

Preferably the drug loaded, coated resin particles have an average diameter (defined as D50%) of 30-300 micrometer.

PULSED-RELEASE DOSAGE FORMS

An exemplary pulsed-release dosage forms may provide at least a part of the dose with a pulsed delayed-release of the drug and another part of the formulation with rapid or immediate-release. The immediate and pulsed delayed-release of the drug can be achieved according to different principles, such as by single dose layered pellets or tablets, by multiple dose layered pellets or tablets, or by two or more different fractions of single or multiple dose layered pellets or tablets, optionally in combination with pellets or tablets having instant-release. Multiple dose layered pellets may be filled into a capsule or together with tablet excipients compressed into a multiple unit tablet. Alternatively, a multiple dose layered tablet may be prepared.

Single dose layered pellets or tablets giving one single delayed-release pulse of galantamine may be prepared. The single dose layered pellets or tablets may comprise a core material, optionally layered on a seed/sphere, the core material comprising galantamine together with a water swellable substance; a surrounding lag time controlling layer, and an outer coating layer positioned to cover the lag time controlling layer. Alternatively, the layered pellets or tablets may comprise a core material comprising the active agent; a surrounding layer comprising a water swellable substance; a surrounding lag time controlling layer; and an outer coating layer positioned to cover the lag time controlling layer.

Multiple dose layered pellets or tablets giving two or more delayed-release pulses of the drug may be prepared comprising a core material, optionally layered on a seed/sphere comprising the active agent and a water swellable substance, a surrounding lag time controlling layer, a layer comprising the active agent optionally together with a water swellable substance; optionally a separating layer which is water-soluble or in water rapidly disintegrating; and an outer coating layer. Alternatively, a multiple dose layered pellets or tablets may comprise a core material, optionally layered on a seed/sphere, comprising the active agent; a surrounding layer comprising a water swellable substance; a surrounding lag time controlling layer; a layer comprising the active agent; optionally a separating layer; and an outer coating layer.

The core material comprising the active agent can be prepared either by coating layering the drug onto a seed, such as for instance sugar spheres, or by extrusion/spheronization of a mixture comprising the drug and pharmaceutically acceptable excipients. It is also possible to prepare the core material by using tablet technology, i.e. compression of drug granules and optionally pharmaceutically acceptable excipients into a tablet core. For pellets of the two types, i.e. single or multiple dose pellets, which have the drug deposited onto a seed/sphere by layering, it is also possible to have an optional layer comprising a water swellable substance beneath the drug containing layer in the core material. The seeds/spheres can be water insoluble and comprise different oxides, celluloses, organic polymers and other materials, alone or in mixtures, or be water soluble and comprise different inorganic salts, sugars and other materials, alone or in mixtures. Further, the seeds/spheres may comprise active agent in the form of crystals, agglomerates, compacts etc. The size of the seeds may be about 0.1 to about 2 mm. Before the seeds are layered, the active substance may be mixed with further components to obtain preferred handling and processing properties and a suitable concentration of the active substance in the final mixture.

Optionally an osmotic agent is placed in the core material. Such an osmotic agent is water soluble and will provide an osmotic pressure in the tablet. Examples of osmotic agents are magnesium sulfate, sodium chloride, lithium chloride, potassium chloride, potassium sulfate, sodium carbonate, lithium sulfate, calcium bicarbonate, sodium sulfate, calcium lactate, urea, magnesium succinate, sucrose or mixtures thereof.

Water swellable substances suitable for the dosage forms are compounds which are able to expand when they are exposed to an aqueous solution, such as gastro-intestinal fluid. One or more water swellable substances may be present in the core material together with the active agent and optionally pharmaceutically acceptable excipient(s). Alternatively, one or more water swellable substances are included in a swelling layer applied onto the core material. As a further alternative, swellable substances(s) they may also be present in an optional swelling layer situated beneath the drug containing layer, if a layered seed or sphere is used as the core material.

The amount of water swellable substance(s) in the swelling layer or in the core to material ratio is chosen in such a way that the core material or the swelling layer in contact with an aqueous solution, such as gastro-intestinal fluid, will expand to such a degree that the surrounding lag-time controlling membrane ruptures. A water swellable substance may also be included in the drug comprising layer of the multiple layered pellets or tablets to increase dissolution rate of the drug fraction.

Suitable substances which can be used as water swellable substances include, for example, low-substituted hydroxypropyl cellulose, e.g. L-HPC; cross-linked polyvinyl pyrrolidone (PVP-XL), e.g. Kollidon® CL and Polyplasdone® XL; cross-linked sodium carboxymethylcellulose, e.g. Ac-di-sol®, Primellose®; sodium starch glycolate, e.g. Primojel®; sodium carboxymethylcellulose, e.g. Nymcel ZSB10®; sodium carboxymethyl starch, e.g. Explotab®; ion-exchange resins, e.g. Dowex® or Amberlite®; microcrystalline cellulose, e.g. Avicel®; starches and pregelatinized starch, e.g. Starch 1500®, Sepistab ST200®; formalin-casein, e.g. Plas-Vita®, and combinations comprising one or more of the foregoing water swellable substances.

The lag time controlling layer is a semipermeable membrane comprising a water resistant polymer that is semipermeable for an aqueous solution, such as gastro-intestinal fluid. Suitable polymers are cellulose acetate, ethylcellulose, polyvinyl acetate, cellulose acetate butyrate, cellulose acetate propionate, acrylic acid copolymers, such as Eudragit® RS or RL, and combinations comprising one or more of the foregoing polymers. The polymer may optionally comprise pore forming agents, such as a water soluble substance, e.g. sucrose, salt; or a water soluble polymer e.g., polyethylene glycol. Also pharmaceutically acceptable excipients such as fillers and membrane strength influencing agents such as talc, aerosil, and/or sodium aluminum silicate may be included.

There is at least one lag time controlling layer present in the dosage form. The lag time controlling layer positioned nearest the inner core material is constructed in the form of a semipermeable membrane that will disrupt after a desired time after ingestion. A desired lag time may be adjusted by the composition and thickness of the layer. The amount of substances forming such a disrupting semipermeable membrane, i.e. a lag time controlling layer, may be about 0.5 to about 25% of the weight of the core material including swelling substances or a swelling layer, preferably about 2 to about 20% by weight.

The lag time controlling layer may comprise a mixture of ethylcellulose and talc. The mixture contains most preferably 10 to 80% w/w of talc.

Before applying the outer coating layer onto the layered pellets or tablets, they may optionally be covered with one or more separating layers comprising excipients. This separating layer separates the composition of the layered pellets or tablets from the outer enteric coating layer. Suitable materials for the optional separating layer are pharmaceutically acceptable compounds such as, for instance, sugar, polyethylene glycol, polyvinyl pyrrolidone, polyvinyl alcohol, polyvinyl acetate, hydroxypropyl cellulose, methylcellulose, ethylcellulose, hydroxypropyl methylcellulose, carboxymethylcellulose sodium and others, and combinations comprising one or more of the foregoing materials. Other additives may also be included into the separating layer.

When the optional separating layer is applied to the layered pellets or tablets it may constitute a variable thickness. The maximum thickness of the optional separating layer is normally only limited by processing conditions. The separating layer may serve as a diffusion barrier and may act as a pH-buffering zone. The optional separating layer may improve the chemical stability of the active substance and/or the physical properties of the dosage form.

Finally the layered pellets or tablets are covered by one or more outer coating layers by using a suitable coating technique. The outer coating layer material may be dispersed or dissolved in either water or in suitable organic solvents. Suitable methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethyl ethylcellulose, shellac or other suitable coating layer polymer(s).

The applied polymer containing layers, and specially the outer coating layers may also contain pharmaceutically acceptable plasticizers to obtain desired mechanical properties.

In one embodiment, the oral dosage formulation may comprise galantamine, or a pharmaceutically acceptable salt thereof, in a controlled-release form, wherein the formulation provides a first maximum plasma concentration of the galantamine (Cₘₐₓ₁) between 0 hours and about 12 hours after administration, and a second maximum plasma concentration or the galantamine (Cₘₐₓ₂) between about 12 hours and about 24 hours after administration at steady-state. The active agent in preferably galantamine hydrobromide.

The formulation further exhibits a first maximum galantamine plasma concentration (Cₘₐₓ₁) between 0 hours and about 12 hours after administration, a second maximum galantamine plasma concentration (Cₘₐₓ₂) between about 12 hours and about 24 hours after administration, and an galantamine plasma concentration at about 24 hours after administration (C₂₄), wherein the average galantamine plasma concentration between about Cₘₐₓ₁ and about Cₘₐₓ₂ is substantially equal to the average galantamine plasma concentration between about Cₘₐₓ₂ and about C₂₄.

Also within this embodiment, the formulation provides a first maximum galantamine plasma concentration (Cₘₐₓ₁) and a first minimum galantamine plasma concentration (Cₘᵢₙ₁) between 0 hours and about 12 hours after administration, a second maximum galantamine plasma concentration (Cₘₐₓ₂), and a galantamine plasma concentration at about 24 hours after administration (C₂₄), wherein the ratio of Cₘₐₓ₁ to Cₘᵢₙ₁ is less than about 4:1 or the ratio of Cₘₐₓ₂ to C₂₄ is less than about 4:1. Preferably the ratio of Cₘₐₓ₁ to Cₘᵢₙ₁ is less than about 2:1. The Cₘₐₓ₂ may occur at about 12 to about 14 hours after administration

Preferably, the pulse-release formulation has, at steady state, a difference between the ratio of Cₘₐₓ₁ to Cₘᵢₙ₁ and the ratio of Cₘₐₓ₂ to C₂₄ of less than about 50%, preferably less than about 40%, and more preferably less than about 30%.

In a preferred embodiment, the sustained-release oral dosage formulation, comprises a first subunit wherein the first subunit comprises a galantamine and a first release-retarding material; and a second subunit, wherein the second subunit comprises galantamine and a second release-retarding material, wherein the first and second release-retarding material can be the same or different, and wherein the dosage formulation, at steady-state, provides a maximum galantamine plasma concentration (Cₘₐₓ) and an galantamine plasma concentration at about 24 hours after administration (C₂₄), wherein the ratio of Cₘₐₓ to C₂₄ is less than about 4:1.

The sustained oral release dosage formulations may optionally comprise an enteric coating as described herein below.

Exemplary Formulations

The various release properties described above may be achieved in a variety of different ways. Suitable formulations include, for example, wax formulations, press coat formulations, easily administered formulations, osmotic pump dosage forms, etc.

Wax Formulations

A wax formulation is a solid dosage form comprising the active agent or a pharmaceutically acceptable salt thereof, most preferably galantamine hydrobromide, in a waxy matrix. The waxy matrix may be prepared by hot melting a suitable wax material and using the melt to granulate the active agent material. The matrix material comprises the waxy material and the active agent.

The wax material can be, for example, an amorphous wax, an anionic wax, an anionic emulsifying wax, a bleached wax, a carnauba wax, a cetyl esters wax, a beeswax, a castor wax, a cationic emulsifying wax, a cetrimide emulsifying wax, an emulsifying wax, a glyceryl behenate, a microcrystalline wax, a nonionic wax, a nonionic emulsifying wax, a paraffin, a petroleum wax, a spermaceti wax, a white wax, a yellow wax, and combinations comprising one or more of the foregoing waxes. These and other suitable waxes are known to those of skill in the art. A cetyl esters wax, for example, preferably has a molecular weight of about 470 to about 490 and is a mixture containing primarily esters of saturated fatty alcohols and saturated fatty acids. The wax material can comprise a carnauba wax, glyceryl behenates, castor wax, and combinations comprising one or more of the foregoing waxes. When the waxy material consists of carnauba wax and no other waxy material is used, the matrix is preferably coated with a functional coating. When the waxy material includes glyceryl behenates and carnauba wax, the matrix can be used without a coating, but may have either a cosmetic coating or a functional coating depending on the precise release profile and appearance desired.

The wax material can be used at about 16% to about 35%, preferably about 20% to about 32%, more preferably about 24% to about 31%, and most preferably about 28% to about 29% of the total weight of the matrix material. When a combination of wax is used, e.g., carnauba wax and glyceryl behenate, the component waxes can be used in a suitable ratio. Certain formulations include the wax material component from 100 to about 85 parts camauba wax and from 0 to about 15 parts glyceryl behenate. In formulations that have a combination of camauba wax and castor wax, for example, the wax component may have about 100 to about 85 parts carnauba wax and 0 to about 15 parts castor wax. When carnauba wax, glyceryl behenate and castor wax are present, the carnauba wax can comprise at least about 85% of the waxy material and the balance of the waxy material is made up of a combination of glyceryl behenate and castor wax, in a suitable relative proportion.

Optionally, fatty acids and fatty acid soaps can be present in the waxy dosage form. In some cases, the fatty acids and/or fatty acid soaps can replace a portion of the wax or waxes. These optional fatty acids and fatty acid soaps can be those that are generally used in the pharmaceutical industry as tableting lubricants, such as, for example, solid fatty acids (for example fatty acids having from about 16 to about 22 carbon atoms), and the alkaline earth metal salts thereof, particularly the magnesium and calcium salts, and combinations comprising one or more of the foregoing fatty acids. The fatty acid can be, for example, stearic acid. The optional fatty acids and fatty acid soaps, when present, can be used in amounts of up to about 10% of the total weight of the matrix material, or about 2.5% to about 9%, or about 2.7% to about 8.6%, or from about 3% to about 6% of the total weight of the matrix material. An amount of up to about 2% of the total core formulation of the optional fatty acid materials may be used as a blend with the melt granulate. Amounts of at least about 1% may be used in this fashion with the remainder being added to the waxes for melting and granulating the active agent.

To prepare the dosage form, the waxes may be melted and used to granulate the active agent. The granulate may be allowed to cool and then be milled to a proper size. Advantageously, the granulate is milled to an average particle size of about 75 microns to about 850 microns, preferably about 150 microns to about 425 microns. The milled granulate may be mixed with optional processing aids. The processing aids include, for example, hydrophobic colloidal silicon dioxide (such as CAB-O-SIL® M5). Hydrophobic silicon dioxide may be used in amounts of less than or equal to about 0.5%, but individual formulations can be varied as required. The blend of the waxy granulate and the processing aids, if any, may be compressed and then optionally coated.

The wax dosage form can include, for example, compressed coated or uncoated tablets, compressed pellets contained in capsules, or loose powder or powder filled capsules.

Press Coat Formulations

A press coat oral dosage form of active agent or a pharmaceutically acceptable salt thereof comprises a core composition and a coating composition press-coated on the core. The core composition comprises a waxy material and active agent or its salt and the coating composition comprises a hydrophilic polymer and optionally active agent or its salt. Preferably the active agent is in the form of galantamine hydrobromide.

The core composition of the press coat dosage from comprises a waxy material. The waxy material can be a hydrophobic waxy material to provide controlled-release of the active agent. In pharmaceutical and/or veterinary products, for example, such waxy materials may be, for example, carnauba wax, tribehenin, fatty alcohols (particularly those having 12-24 carbon atoms, such as lauryl alcohol, myristyl alcohol, stearyl alcohol, palmityl alcohol, etc.), fatty acids (particularly those having 12-24 carbon atoms, such as lauric acid, myristic acid, stearic acid, palmitic acid, etc), polyethylenes, castor wax, C₁₆₋₃₀ fatty acid triglycerides, beeswax, and combinations comprising one or more of the foregoing waxes.

The coating composition comprises a hydrophilic polymer. The hydrophilic polymer can provide for controlled-release of the active agent. The hydrophilic polymer providing controlled-release may be a film forming polymer, such as a hydrophilic cellulose polymer. Such a hydrophilic cellulose polymer may be hydroxyalkyl cellulose polymer, for example hydroxyethylcellulose (HEC), hydroxypropyl cellulose (HPC), hydroxypropylmethylcellulose (HPMC), hydroxypropylethylcellulose (HPEC), hydroxypropylpropylcellulose (HPPC), hydroxypropylbutylcellulose (HPBC), and combinations comprising one or more of the foregoing polymers.

Both the core composition and the coating composition may further include a filler, such as a water insoluble filler, water soluble filler, and mixtures thereof. A water-insoluble filler can be talc or a calcium salt such as a calcium phosphate, e.g., a dicalcium phosphate. The filler in the coating composition can be the same or different as the filler in the core composition, if any. For example, the core composition can include a water-soluble filler while the coating composition can include a water-insoluble filler.

Optional excipients can also be present in the core composition and the coating composition, including lubricants (such as talc and magnesium stearate), glidants (such as fumed or colloidal silica), pH modifiers (such as acids, bases and buffer systems), pharmaceutically useful processing aids, and combinations comprising one or more of the foregoing excipients. Excipients in the coating composition can be the same or different as those in the core composition.

In the formation of a dosage form, the core composition can be press-coated with the press-coat composition coating formulation to form a tablet. The tablet can be further coated with optional additional coatings. The additional coatings can be pH-dependent or pH-independent, aesthetic or functional, and can include the active agent in immediate or controlled-release. The optional additional coating can include an active agent, either active agent or a pharmaceutically active salt thereof or a different active agent than is contained in the core composition and the coating composition. The additional coating may, for example, include an immediate-release dosage form of active agent.

The press coat formulations may have substantially zero order, first order, and second order release rate profiles by adjusting the amount of active agent in the core composition and the coating composition. The ratio of the active agent in the core composition (Core_{AA}) to active agent in the coating composition (Coat_{AA}) may be about 1:99 to about 99:1, more preferably about 95:5 to about 5:99, most preferably about 9:1 to about 1:9. For the highly soluble active agents, including active agent and other highly soluble active agents that may be used in combination with active agent, a Core_{AA}:Coat_{AA} of about 3:4 to about 5:3 is can provide a substantially zero order release rate, a Core_{AA}:Coat_{AA} of less than about 3:4 can provide a substantially first order release rate, and a Core_{AA}:Coat_{AA} of greater than about 5:3 can provide a substantially second order release rate.

In forming the dosage form, the core composition components (active agent, wax, and optional excipients) are blended together and compressed into suitable cores. The blending can take place in a suitable order of addition. The cores may be blended by starting with the smallest volume component and then successively adding the larger volume components. Another process is to melt the wax and to blend the active agent and optional excipients into the melted wax. Alternatively, the active agent, wax and optional excipients can be blended together and then subjected to a temperature at which the wax will melt. Once cooled, the solidified mass can be milled into granules for compaction into cores.

The press coat formulations can be 5 mg, 10 mg, 25 mg, and 50 mg tablets press coated tablets. One exemplary press coat active agent formulation comprises 25 mg active agent in an immediate-release coating composition and 22.5 mg active agent between the core composition and the coating composition. In this example, the 0-4 hour cumulative release of active agent in 0.1 N hydrochloric acid is may be at least about 25% to about 50%, more preferably about 35 to about 40%, of the loaded dose, and the 0-12 hour cumulative release of the active agent in 0.1 N hydrochloric acid may be at least about 75%, more preferably at least about 85%, of the dosage form dose. In another example, a 50 mg active agent formulation comprises a 3:2:1 (core:press coat:immediate-release coat) ratio, e.g., a core composition comprising 25 mg of active agent, a coating composition comprising 10 mg of active agent, and an immediate-release loading dose comprising 5 mg of active agent.

Easily Administered Dosage Forms

Chewable Tablets

Another solid dosage form is a chewable tablet containing the active agent. A chewable tablet comprises a chewable base and optionally a sweetener. The chewable base comprises an excipient such as, for example, mannitol, sorbitol, lactose, or a combination comprising one or more of the foregoing excipients. The optional sweetener used in the chewable dosage form may be, for example, digestible sugars, sucrose, liquid glucose, sorbitol, dextrose, isomalt, liquid maltitol, aspartame, lactose, and combinations comprising one or more of the foregoing sweeteners. In certain cases, the chewable base and the sweetener may be the same component. The chewable base and optional sweetener may comprise about 50 to about 90 weight % of the total weight of the dosage form.

The chewable dosage form may additionally contain preservatives, agents that prevent adhesion to oral cavity and crystallization of sugars, flavoring agents, souring agents, coloring agents, and combinations comprising one or more of the foregoing agents. Glycerin, lecithin, hydrogenated palm oil or glyceryl monostearate may be used as a protecting agent of crystallization of the sugars in an amount of about 0.04 to about 2.0 weight % of the total weight of the ingredients, to prevent adhesion to oral cavity and improve the soft property of the products. Additionally, isomalt or liquid maltitol may be used to enhance the chewing properties of the chewable dosage form.

A method of making a chewable dosage form of the active agent is similar to the method used to make soft confectionary. The method generally involves the formation of a boiled sugar-digestible sugar blend to which is added a frappe mixture. The boiled sugar-digestible sugar blend may be prepared from sugar and digestible sugar blended in parts by weight ratio of 90:10 to 10:90. This blend may be heated to temperatures above 250° F. to remove water and to form a molten mass. The frappe mixture may be prepared from gelatin, egg albumen, milk proteins such as casein, and vegetable proteins such as soy protein, and the like which are added to a gelatin solution and rapidly mixed at ambient temperature to form an aerated sponge like mass. The frappe mixture is then added to the molten candy base and mixed until homogenous at temperatures between 150° F. to about 250° F. A wax matrix containing the active agent may then be added as the temperature of the mix is lowered to about 120° F. to about 194° F., whereupon additional ingredients such as flavors, colorants, and preservatives may be added. The formulation is further cooled and formed to pieces of desired dimensions.

Fast Dissolving Formulations

Another oral dosage form is a non-chewable, fast dissolving dosage form of the active agent. These dosage forms can be made by methods known to those of ordinary skill in the art of pharmaceutical formulations. For example, Cima Labs has produced oral dosage forms including microparticles and effervescents which rapidly disintegrate in the mouth and provide adequate taste-masking. Cima Labs has also produced a rapidly dissolving dosage form containing the active agent and a matrix that includes a nondirect compression filler and a lubricant. Zydis (ZYPREXA) is produced by Eli Lilly as in a rapidly dissolvable, freeze-dried, sugar matrix formulated as a rapidly dissolving tablet. U.S. Pat. No. 5,178,878 and U.S. Pat. No. 6,221,392 provide teachings regarding fast-dissolve dosage forms.

An exemplary fast dissolve dosage form includes a mixture incorporating a water and/or saliva activated effervescent disintegration agent and microparticles. The microparticles incorporate an active agent together with a protective material substantially encompassing the active agent. The term “substantially encompassing” as used in this context means that the protective material substantially shields the active agent from contact with the environment outside of the microparticle. Thus, each microparticle may incorporate a discrete mass of the active agent covered by a coating of the protective material, in which case the microparticle can be referred to as a “microcapsule”. Alternatively or additionally, each microparticle may have the active agent dispersed or dissolved in a matrix of the protective material. The mixture including the microparticles and effervescent agent desirably may be present as a tablet of a size and shape adapted for direct oral administration to a patient, such as a human patient. The tablet is substantially completely disintegrable upon exposure to water and/or saliva. The effervescent disintegration agent is present in an amount effective to aid in disintegration of the tablet, and to provide a distinct sensation of effervescence when the tablet is placed in the mouth of a patient.

The effervescent sensation is not only pleasant to the patient but also tends to stimulate saliva production, thereby providing additional water to aid in further effervescent action. Thus, once the tablet is placed in the patient's mouth, it will disintegrate rapidly and substantially completely without any voluntary action by the patient. Even if the patient does not chew the tablet, disintegration will proceed rapidly. Upon disintegration of the tablet, the microparticles are released and can be swallowed as a slurry or suspension of the microparticles. The microparticles thus may be transferred to the patient's stomach for dissolution in the digestive tract and systemic distribution of the pharmaceutical ingredient.

The term effervescent disintegration agent(s) includes compounds which evolve gas. The preferred effervescent agents evolve gas by means of chemical reactions which take place upon exposure of the effervescent disintegration agent to water and/or to saliva in the mouth. The bubble or gas generating reaction is most often the result of the reaction of a soluble acid source and an alkali metal carbonate or carbonate source. The reaction of these two general classes of compounds produces carbon dioxide gas upon contact with water included in saliva.

Such water activated materials should be kept in a generally anhydrous state with little or no absorbed moisture or in a stable hydrated form since exposure to water will prematurely disintegrate the tablet. The acid sources or acid may be any which are safe for human consumption and may generally include food acids, acid anhydrides and acid salts. Food acids include citric acid, tartaric acid, malic acid, fumaric acid, adipic acid, and succinic acids etc. Because these acids are directly ingested, their overall solubility in water is less important than it would be if the effervescent tablet formulations of the present invention were intended to be dissolved in a glass of water. Acid anhydrides and acid of the above described acids may also be used. Acid salts may include sodium, dihydrogen phosphate, disodium dihydrogen pyrophosphate, acid citrate salts and sodium acid sulfite.

Carbonate sources include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate, amorphous calcium carbonate, and combinations comprising one or more of the foregoing carbonates.

The effervescent disintegration agent is not always based upon a reaction which forms carbon dioxide. Reactants which evolve oxygen or other gasses which are pediatrically safe are also considered within the scope. Where the effervescent agent includes two mutually reactive components, such as an acid source and a carbonate source, it is preferred that both components react substantially completely. Therefore, an equivalent ratio of components which provides for equal equivalents is preferred. For example, if the acid used is diprotic, then either twice the amount of a mono-reactive carbonate base, or an equal amount of a di-reactive base should be used for complete neutralization to be realized. However, the amount of either acid or carbonate source may exceed the amount of the other component. This may be useful to enhance taste and/or performance of a tablet containing an overage of either component. In this case, it is acceptable that the additional amount of either component may remain unreacted.

In general, the amount of effervescent disintegration agent useful for the formation of tablets is about 5 to about 50% by weight of the final composition, preferably about 15 and about 30% by weight thereof, and most preferably about 20 and about 25% by weight of the total composition.

More specifically, tablets according to the present invention should contain an amount of effervescent disintegration agent effective to aid in the rapid and complete disintegration of the tablet when orally administered. By “rapid”, it is understood that the tablets should disintegrate in the mouth of a patient in less than 10 minutes, and desirably between about 30 seconds and about 7 minutes, preferably the tablet should dissolve in the mouth between about 30 seconds and about 5 minutes. Disintegration time in the mouth can be measured by observing the disintegration time of the tablet in water at about 37° C. The tablet is immersed in the water without forcible agitation. The disintegration time is the time from immersion for substantially complete dispersion of the tablet as determined by visual observation. As used herein, the term “complete disintegration” of the tablet does not require dissolution or disintegration of the microcapsules or other discrete inclusions.

The active agent is present in microparticles. Each microparticle incorporates the active agent in conjunction with a protective material. The microparticle may be provided as a microcapsule or as a matrix-type microparticle. Microcapsules may incorporate a discrete mass of the active agent surrounded by a discrete, separately observable coating of the protective material. Conversely, in a matrix-type particle, the active agent is dissolved, suspended or otherwise dispersed throughout the protective material. Certain microparticles may include attributes of both microcapsules and matrix-type particle. For example, a microparticle may incorporate a core incorporating a dispersion of the active agent in a first protective material and a coating of a second protective material, which may be the same as or different from the first protective material surrounding the core. Alternatively, a microparticle may incorporate a core consisting essentially of the active agent and a coating incorporating the protective material, the coating itself having some of the pharmaceutical ingredient dispersed within it.

The microparticles may be about 75 and 600 microns mean outside diameter, and more preferably between about 150 and about 500 microns. Microparticles above about 200 microns may be used. Thus, the microparticles may be between about 200 mesh and about 30 mesh U.S. standard size, and more preferably between about 100 mesh and about 35 mesh.

Tablets according can be manufactured by well-known tableting procedures. In common tableting processes, the material which is to be tableted is deposited into a cavity, and one or more punch members are then advanced into the cavity and brought into intimate contact with the material to be pressed, whereupon compressive force is applied. The material is thus forced into conformity with the shape of the punches and the cavity. Hundreds, and even thousands, of tablets per minute can be produced in this fashion.

Another exemplary fast-dissolve dosage form is a hard, compressed, rapidly dissolvable dosage form adapted for direct oral dosing. The dosage form includes an active agent often in the form of a protected particle, and a matrix. The matrix includes a nondirect compression filler and a lubricant, although, it may include other ingredients as well. The dosage form is adapted to rapidly dissolve in the mouth of a patient, yet it has a friability of about 2% or less when tested according to the USP. Generally, the dosage form will also have a hardness of at least about 15 to about 20 Newtons (about 1.53-2.04 kilopond (kp). Not only does the dosage form dissolve quickly, it does so in a way that provides a positive organoleptic sensation to the patient. In particular, the dosage form dissolves with a minimum of unpleasant grit which is tactilely inconsistent with a positive organoleptic sensation to the patient.

The protective materials may include a polymers conventionally utilized in the formation of microparticles, matrix-type microparticles and microcapsules. Among these are cellulosic materials such as naturally occurring cellulose and synthetic cellulose derivatives; acrylic polymers and vinyl polymers. Other simple polymers include proteinaceous materials such as gelatin, polypeptides and natural and synthetic shellacs and waxes. Protective polymers may also include ethylcellulose, methylcellulose, carboxymethyl cellulose and acrylic resin material sold under the registered trademark EUDRAGIT by Rohm Pharma GmbH of Darmstadt, Germany.

Generally, when a coating is used, the coating may be used at greater than or equal to about 5 percent based on the weight of the resulting particles. More preferable, the coating should constitute at least about 10 percent by weight of the particle. The upper limit of protective coating material used is generally less critical, except that where a rapid-release of the active ingredient is desired, the amount of coating material should not be so great that the coating material impedes the release profile of the active agent or pharmaceutical ingredient when ingested. Thus, it may be possible to use greater than 100 percent of the weight of the core, thereby providing a relatively thick coating.

The filler comprises a nondirect compression fillers. Exemplary fillers include, for example, nondirect compression sugars and sugar alcohols which meet the specifications discussed above. Such sugars and sugar alcohols include, without limitation, dextrose, mannitol, sorbitol, lactose and sucrose. Of course, dextrose, for example, can exist as either a direct compression sugar, i.e., a sugar which has been modified to increase its compressibility, or a nondirect compression sugar.

Generally, the balance of the formulation can be matrix. Thus the percentage of filler can approach 100% by weight. However, generally, the amount of nondirect compression filler is about 25 to about 95%, preferably about 50 and about 95% and more preferably about 60 to about 95%.

In the fast-dissolve dosage form, a relatively high proportion of lubricant should be used when compared to the prior art. Lubricants, and in particular, hydrophobic lubricants such as magnesium stearate, are generally used in an amount of about 0.25 to about 5%, according to the Handbook of Pharmaceutical Excipients. It has been found that the amount of lubricant used can be double, triple or even quadruple that proposed previously. Specifically, the amount of lubricant used can be about 1 to about 2.5% by weight, and more preferably about 1.5 to about 2% by weight. Despite the use of this relatively high rate of lubricant, the formulations exhibit a superior compressibility, hardness, and rapid dissolution within the mouth.

Hydrophobic lubricants include, for example, alkaline stearates, stearic acid, mineral and vegetable oils, glyceryl behenate, sodium stearyl fumarate, and combinations comprising one or more of the foregoing lubricants. Hydrophilic lubricants can also be used.

The dosage forms may have a hardness of at least about 15 Newtons (about 1.53 kp) and are designed to dissolve spontaneously and rapidly in the mouth of a patient in less than about 90 seconds to thereby liberate the particles. Preferably the dosage form will dissolve in less than about 60 seconds and even more preferably about 45 seconds. This measure of hardness is based on the use of small tablets of less than about 0.25 inches in diameter. A hardness of at least about 20 Newtons (about 2.04 kp) is preferred for larger tablets. Direct compression techniques are preferred for the formation of the tablets.

In one embodiment, the galantamine fast dissolve solid dosage form comprises a pharmaceutically acceptable carrier, wherein the carrier is substantially free of a spray dried mixture of lactose monohydrate and microcrystalline cellulose. The formulation preferably exhibits a dissolution profile such that after 0.5 hour at least about 80% of the galantamine or galantamine salt is released in 500 ml of purified water at 37° C. in Apparatus 2 (USP, <711 > Dissolution, paddle, 50 rpm).

The fast dissolve solid dosage formulation may comprise the active agent and carrier in the form of particles having a particle size distribution that allows for the ease of processing the material into tablets, by direct compression techniques for example, without segregation of the excipients. The desired particle range of active agent and excipient may be obtained by processes known in the art, including granulating, screening, milling, and the like.

Sprinkle Dosage Forms

Sprinkle dosage forms include particulate or pelletized forms of the active agent, optionally having functional or non-functional coatings, with which a patient or a caregiver can sprinkle the particulate/pelletized dose into drink or onto soft food. A sprinkle dosage form may comprise particles of about 10 to about 100 micrometers in their major dimension. Sprinkle dosage forms may be in the form of optionally coated granules or as microcapsules. Sprinkle dosage forms may be immediate or controlled-release formulations such as sustained-release formulations. See U.S. Pat. No. 5,084,278, which is hereby incorporated by reference for its teachings regarding microcapsule formulations, which may be administered as sprinkle dosage forms.

Taste Masked Solid Dosage Forms

A solid oral dosage form may comprise a taste-masked dosage form. The taste-masked dosage forms may be liquid dosage forms such as those disclosed by F. H. Faulding, Inc. (U.S. Pat. No. 6,197,348).

A solid taste masked dosage form comprises a core element comprising the active agent and a coating surrounding the core element. The core element comprising the active agent may be in the form of a capsule or be encapsulated by micro-encapsulation techniques, where a polymeric coating is applied to the formulation. The core element includes the active agent and may also include carriers or excipients, fillers, flavoring agents, stabilizing agents and/or colorants.

The taste masked dosage form may include about 77 weight % to about 100 weight %, preferably about 80 weight % to about 90 weight %, based on the total weight of the composition of the core element including the active agent; and about 20 weight % to about 70 weight %, of a substantially continuous coating on the core element formed from a coating material including a polymer. The core element includes about 52 to about 85% by weight of the active agent; and approximately 5% to about 25% by weight of a supplementary component selected from waxes, water insoluble polymers, enteric polymers, and partially water soluble polymers, other suitable pharmaceutical excipients, and combinations comprising one or more of the foregoing components.

The core element optionally include carriers or excipients, fillers, flavoring agents, stabilizing agents, colorants, and combinations comprising one or more of the foregoing additives. Suitable fillers include, for example, insoluble materials such as silicon dioxide, titanium dioxide, talc, alumina, starch, kaolin, polacrilin potassium, powdered cellulose, and microcrystalline cellulose, and combinations comprising one or more of the foregoing fillers. Soluble fillers include, for example, mannitol, sucrose, lactose, dextrose, sodium chloride, sorbitol, and combinations comprising one or more of the foregoing fillers. The filler may be present in amounts of up to about 75 weight % based on the total weight of the composition. The particles of the core element may be in the range of the particle size set forth above for core particles of core elements.

The core element may be in the form of a powder, for example, having a particle size range of about 35 μm to about 125 μm. The small particle size facilitates a substantially non-gritty feel in the mouth. Small particle size also minimizes break-up of the particles in the mouth, e.g. by the teeth. When in the form of a powder, the taste masked dosage form may be administered directly into the mouth or mixed with a carrier such as water, or semi-liquid compositions such as yogurt, and the like. However, the taste masked active agent may be provided in any suitable unit dosage form.

The coating material of the taste-masked formulation may take a form which provides a substantially continuous coating and still provides taste masking. In some cases, the coating also provides controlled-release of the active agent. The polymer used in taste masked dosage form coating may be a water insoluble polymer such as, for example, ethyl cellulose. The coating material of the taste masked dosage form may further include a plasticizer.

A method of preparing taste-masked pharmaceutical formulations such as powdered formulations includes mixing a core element and a coating material in a diluent and spray drying the mixture to form a taste-masked formulation. Spray drying of the pharmaceutically active ingredient and polymer in the solvent involves spraying a stream of air into an atomized suspension so that solvent is caused to evaporate leaving the active agent coated with the polymer coating material.

For a solvent such as methylene chloride, the solvent concentration in the drying chamber may be maintained above about 40,000 parts, or about 40,000 to about 100,000 parts per million of organic solvent. The spray-drying process for such solvents may be conducted at a process temperature of about 5° C. to about 35° C. Spray drying of the dosage forms may be undertaken utilizing either rotary, pneumatic or pressure atomizers located in either a co-current, counter-current or mixed-flow spray dryer or variations thereof. The drying gas may be heated or cooled to control the rate of drying. A temperature below the boiling point of the solvent may be used. Inlet temperatures may be about 40° C. to about 120° C. and outlet temperatures about 5° C. to about 35° C.

The coat formation may be optimized to meet the needs of the material or application. Controlling the process parameters including temperature, solvent concentration, spray dryer capacity, atomizing air pressure, droplet size, viscosity, total air pressure in the system and solvent system, allows the formation of a range of coats, ranging from dense, continuous, non-porous coats through to more porous microcapsule/polymer matrices.

A post-treatment step may be used to remove residual solvent. The post treatment may include a post drying step including drying the final product on a tray and drying the product at a bed temperature sufficient to remove excess solvent, but not degrade the active agent. Preferably the drying temperature is in the range of about 35° C. to about 4° C. Once completed, the product may be collected by a suitable method, such as collection by sock filters or cyclone collection.

Taste Masked Liquid Dosage Forms

Liquid dosage forms of the active agent may be formulated that also provide adequate taste masking. A taste masked liquid dosage form can comprise a suspension of microcapsules taste masked as a function of the pH of a suspending medium and a polymer coating. Many active agents are less soluble at higher or lower pH than at the pH value of the mouth, which is around 5.9. In these cases, the active agent can be insufficiently solubilized to be tasted if the equilibrium concentration is below the taste threshold. However, problems can arise if all of the suspended particles are not swallowed because the active agent which remains in the mouth is able to dissolve at the pH of the mouth. The use of polymeric coatings on the active agent particles, which inhibit or retard the rate of dissolution and solubilization of the active agent is one means of overcoming the taste problems with delivery of active agents in suspension. The polymeric coating allows time for all of the particles to be swallowed before the taste threshold concentration is reached in the mouth.

Optimal taste masked liquid formulations may be obtained when consideration is given to: (i) the pH of maximum insolubility of the active agent; (ii) the threshold concentration for taste of the active agent; (iii) the minimum buffer strength required in the medium to avoid delayed or after taste; (iv) the pH limit beyond which further increase or decrease of pH leads to unacceptable instability of the active agent; and (v) the compatibility and chemical, physical and microbial stability of the other ingredients to the pH values of the medium.

A taste masked liquid dosage form thus comprises the active agent, a polymer with a quaternary ammonium functionality encapsulating the active agent, and a suspending medium adjusted to a pH at which the active agent remains substantially insoluble, for suspending the encapsulated active agent. The active agent is taste masked by the combination of the polymer and suspending medium.

The active agent may be in the form of its neutral or salt form and may be in the form of particles, crystals, microcapsules, granules, microgranules, powders, pellets, amorphous solids or precipitates. The particles may further include other functional components. The active agent may have a defined particle size distribution, preferably in the region of about 0.1 to about 500 μm, more preferably about 1 to about 250 μm, and most preferably about 10 to about 150 μm, where there is acceptable mouth feel and little chance of chewing on the residual particles and releasing the active agent to taste.

The taste masked liquid dosage form may include, along with the active agent, other functional components present for the purpose of modifying the physical, chemical, or taste properties of the active agent. For example the active agent may be in the form of ion-exchange or cyclodextrin complexes or the active agent may be included as a mixture or dispersion with various additives such as waxes, lipids, dissolution inhibitors, taste-masking or -suppressing agents, carriers or excipients, fillers, and combinations comprising one or more of the foregoing components.

The polymer used to encapsulate the pharmaceutically active ingredient or the pharmaceutical unit is preferably a polymer having a quaternary ammonium functionality, i.e., a polymer having quaternary ammonium groups on the polymer backbone. These polymers are more effective in preventing the taste perception of the active agent when the resulting microcapsules are formulated as suspensions and stored for long periods despite their widely recognized properties of being permeable to water and dissolved active agents. A suitable polymer is a copolymer of acrylic and methacrylic acid esters with quaternary ammonium groups. The polymer may be a copolymer of methyl methacrylate and triethylammonium methacrylate. Specific examples of suitable polymer include EUDRAGIT RS or EUDRAGIT RL, available from Rohm America, LLC, Piscataway, N.J. used individually or in combination to change the permeability of the coat. A polymer coat having a blend of the RS or RL polymer along with other pharmaceutically acceptable polymers may also be used. These other polymers may be cellulose ethers such as ethyl cellulose, cellulose esters such as cellulose acetate and cellulose propionate, polymers that dissolve at acidic or alkaline pH, such as EUDRAGIT E, cellulose acetate phthalate, and hydroxypropylmethyl cellulose phthalate.

The quantity of polymer used in relation to the active agent is about 0.01-10:1, preferably about 0.02-1:1, more preferably about 0.03-0.5:1 and most preferably about 0.05-0.3:1 by weight.

The pharmaceutically active agent or the active agent particle may be suspended, dispersed or emulsified in the suspending medium after encapsulation with the polymer. The suspending medium may be a water-based medium, but may be a non-aqueous carrier as well, constituted at an optimum pH for the active agent or pharmaceutical unit, such that the active agent remains substantially insoluble. The pH and ionic strength of the medium may be selected on the basis of stability, solubility and taste threshold to provide the optimum taste masking effect, and which is compatible with the stability of the active agent the polymer coat and the coating excipients.

Buffering agents may be included in the suspending medium for maintaining the desired pH. The buffering agents may include dihydrogen phosphate, hydrogen phosphate, amino acids, citrate, acetate, phthalate, tartrate salts of the alkali or alkaline earth metal cations such as sodium, potassium, magnesium, calcium, and combinations comprising one or more of the foregoing buffering agents. The buffering agents may be used in a suitable combination for achieving the required pH and may be of a buffer strength of about 0.01 to about 1 moles/liter of the final formulation, preferably about 0.01 to about 0.1 moles/liter, and most preferably about 0.02 to about 0.05 moles/liter.

The taste masked liquid dosage form may further include other optional dissolved or suspended agents to provide stability to the suspension. These include suspending agents or stabilizers such as, for example, methyl cellulose, sodium alginate, xanthan gum, (poly)vinyl alcohol, microcrystalline cellulose, colloidal silicas, bentonite clay, and combinations comprising one or more of the foregoing agents. Other agents used include preservatives such as methyl, ethyl, propyl and butyl parabens, sweeteners such as sucrose, saccharin sodium, aspartame, mannitol, flavorings such as grape, cherry, peppermint, menthol and vanilla flavors, and antioxidants or other stabilizers, and combinations comprising one or more of the foregoing agents.

A method of preparing a taste masked dosage form for oral delivery, comprises encapsulating the active agent with a polymer having a quaternary ammonium functionality; and adding a suspending medium adjusted to a pH at which the active agent is substantially insoluble, for suspending the encapsulated active agent; wherein the active agent is taste masked by the combination of the polymer and the medium. In the process, the polymer for encapsulation of the active agent or active agent-containing particle is dissolved in a solution or solvent chosen for its poor solubility for the active agent and good solubility for the polymer. Examples of appropriate solvents include but are not limited to methanol, ethanol, isopropanol, chloroform, methylene chloride, cyclohexane, and toluene, either used in combination or used alone. Aqueous dispersions of polymers may also be used for forming the active agent microparticles.

Encapsulation of the active agent or pharmaceutical unit by the polymer may be performed by a method such as suspending, dissolving, or dispersing the pharmaceutically active ingredient in a solution or dispersion of polymer coating material and spray drying, fluid-bed coating, simple or complex coacervation, coevaporation, co-grinding, melt dispersion and emulsion-solvent evaporation techniques, and the like.

The polymer coated active agent powder can also as an alternative be applied for the preparation of reconstitutable powders, i.e., dry powder active agent products that are reconstituted as suspensions in a liquid vehicle such as water before usage. The reconstitutable powders have a long shelf life and the suspensions, once reconstituted, have adequate taste masking.

Osmotic Pump Dosage Forms

Another dosage form of the active agent is one formulated with OROS technology (Alza Corporation, Mountain View, Calif.) also know as an “osmotic pump”. Such dosage forms have a fluid-permeable (semipermeable) membrane wall, an osmotically active expandable driving member (the osmotic push layer), and a density element for delivering the active agent. In an osmotic pump dosage form, the active material may be dispensed through an exit means comprising a passageway, orifice, or the like, by the action of the osmotically active driving member. The active agent of the osmotic pump dosage form may be formulated as a thermo-responsive formulation in which the active agent is dispersed in a thermo-responsive composition. Alternatively, the osmotic pump dosage form may contain a thermo-responsive element comprising a thermo-responsive composition at the interface of the osmotic push layer and the active agent composition.

The osmotic pump dosage form comprises a semipermeable membrane. The capsule or other dispenser of the osmotic pump dosage form can be provided with an outer wall comprising the selectively semipermeable material. A selectively permeable material is one that does not adversely affect a host or animal, is permeable to the passage of an external aqueous fluid, such as water or biological fluids, while remaining essentially impermeable to the passage of the active agent, and maintains its integrity in the presence of a thermotropic thermo-responsive composition, that is it does not melt or erode in its presence. The selectively semipermeable material forming the outer wall is substantially insoluble in body fluids, nontoxic, and non-erodible.

Representative materials for forming the selectively semipermeable wall include semipermeable homopolymers, semipermeable copolymers, and the like. Suitable materials include, for example, cellulose esters, cellulose monoesters, cellulose diesters, cellulose triesters, cellulose ethers, cellulose ester-ethers, and combinations comprising one or more of the foregoing materials. These cellulosic polymers have a degree of substitution, D.S., on their anhydroglucose unit from greater than 0 up to 3 inclusive. By degree of substitution is meant the average number of hydroxyl groups originally present on the anhydroglucose unit that are replaced by a substituting group, or converted into another group. The anhydroglucose unit can be partially or completely substituted with groups such as acyl, alkanoyl, aroyl, alkyl, alkenyl, alkoxy, halogen, carboalkyl, alkylcarbamate, alkylcarbonate, alkylsulfonate, alkylsulfamate, and like semipermeable polymer forming groups.

Other selectively semipermeable materials include, for example, cellulose acylate, cellulose diacylate, cellulose triacylate, cellulose acetate, cellulose diacetate, cellulose triacetate, mono-, di- and tri-cellulose alkanylates, mono-, di- and tri-alkenylates, mono-, di- and tri-aroylates, and the like, and combinations comprising one or more of the foregoing materials. Exemplary polymers including cellulose acetate having a D.S. of 1.8 to 2.3 and an acetyl content of about 32 to about 39.9%; cellulose diacetate having a D.S. of 1 to 2 and an acetyl content of about 21 to about 35%; cellulose triacetate having a D.S of 2 to 3 and an acetyl content of about 34 to about 44.8%, and the like. More specific cellulosic polymers include cellulose propionate having a D.S. of 1.8 and a propionyl content of about 38.5%; cellulose acetate propionate having an acetyl content of about 1.5 to about 7% and an propionyl content of about 39 to about 42%; cellulose acetate propionate having an acetyl content of about 2.5 to about 3%, an average propionyl content of about 39.2 to about 45% and a hydroxyl content of about 2.8 to about 5.4%; cellulose acetate butyrate having a D.S. of 1.8, an acetyl content of about 13 to about 15%, and a butyryl content of about 34 to about 39%; cellulose acetate butyrate having an acetyl content of about 2 to about 29.5%, a butyryl content of about 17 to about 53%, and a hydroxyl content of about 0.5 to about 4.7%; cellulose triacylates having a D.S. of 2.9 to 3 such as cellulose trivalerate, cellulose trilaurate, cellulose tripalmitate, cellulose trioctanoate, and cellulose tripropionate; cellulose diesters having a D.S. of 2.2 to 2.6 such as cellulose disuccinate, cellulose dipalmitate, cellulose dioctanoate, cellulose dicarpylate and the like; mixed cellulose esters such as cellulose acetate valerate, cellulose acetate succinate, cellulose propionate succinate, cellulose acetate octanoate, cellulose valerate palmitate, cellulose acetate heptonate, and the like, and combinations comprising one or more of the foregoing polymers.

Additional selectively semipermeable polymers include, for example, acetaldehyde dimethyl cellulose acetate, cellulose acetate ethylcarbamate, cellulose acetate methylcarbamate, cellulose dimethylaminoacetate, semi-permeable polyamides, semipermeable polyurethanes, semi-permeable polysulfanes, semipermeable sulfonated polystyrenes, cross-linked, selectively semipermeable polymers formed by the coprecipitation of a polyanion and a polycation, selectively semipermeable silicon rubbers, semipermeable polystyrene derivates, semipermeable poly(sodium styrenesulfonate), semipermeable poly(vinylbenzyltrimethyl) ammonium chloride polymers, and combinations comprising one or more of the foregoing polymers.

The osmotically expandable driving member, or osmotic push layer, of the soft capsule osmotic pump dosage form is swellable and expandable inner layer. The materials used for forming the osmotic push layer, are neat polymeric materials, and/or polymeric materials blended with osmotic agents that interact with water or a biological fluid, absorb the fluid, and swell or expand to an equilibrium state. The polymer should exhibit the ability to retain a significant fraction of imbibed fluid in the polymer molecular structure. Such polymers may be, for example, gel polymers that can swell or expand to a very high degree, usually exhibiting about a 2 to 50-fold volume increase. Swellable, hydrophilic polymers, also known as osmopolymers, can be non-cross-linked or lightly cross-linked. The cross-links can be covalent or ionic bonds with the polymer possessing the ability to swell but not dissolve in the presence of fluid. The polymer can be of plant, animal or synthetic origin. Polymeric materials useful for the present purpose include poly(hydroxyalkyl methacrylate) having a molecular weight of about 5,000 to about 5,000,000, poly(vinylpyrrolidone) having a molecular weight of about 10,000 to about 360,000, anionic and cationic hydrogels, poly(electrolyte) complexes, poly(vinyl alcohol) having a low acetate residual, a swellable mixture of agar and carboxymethyl cellulose, a swellable composition comprising methyl cellulose mixed with a sparingly crosslinked agar, a water-swellable copolymer produced by a dispersion of finely divided copolymer of maleic anhydride with styrene, ethylene, propylene, or isobutylene, water swellable polymer of N-vinyl lactams, and the like, and combinations comprising one or more of the foregoing polymers. Other gelable, fluid imbibing and retaining polymers useful for forming the osmotic push layer include pectin having a molecular weight ranging of about 30,000 to about 300,000, polysaccharides such as agar, acacia, karaya, tragacanth, algins and guar, acidic carboxy polymer and its salt derivatives, polyacrylamides, water-swellable indene maleic anhydride polymers; polyacrylic acid having a molecular weight of about 80,000 to about 200,000; POLYOX, polyethylene oxide polymers having a molecular weight of about 100,000 to about 5,000,000, and greater, starch graft copolymers, polyanions and polycations exchange polymers, starch-polyacrylonitrile copolymers, acrylate polymers with water absorbability of about 400 times its original weight, diesters of polyglucan, a mixture of cross-linked polyvinyl alcohol and poly(N-vinyl-2-pyrrolidone), zein available as prolamine, poly(ethylene glycol) having a molecular weight of about 4,000 to about 100,000, and the like, and combinations comprising one or more of the foregoing polymers.

The osmotically expandable driving layer of the osmotic pump dosage form may further contain an osmotically effective compound (osmagent) that can be used neat or blended homogeneously or heterogeneously with the swellable polymer, to form the osmotically expandable driving layer. Such osmagents include osmotically effective solutes that are soluble in fluid imbibed into the swellable polymer, and exhibit an osmotic pressure gradient across the semipermeable wall against an exterior fluid. Suitable osmagents include, for example, solid compounds such as magnesium sulfate, magnesium chloride, sodium chloride, lithium chloride, potassium sulfate, sodium sulfate, mannitol, urea, sorbitol, inositol, sucrose, glucose, and the like, and combinations comprising one or more of the foregoing osmagents. The osmotic pressure in atmospheres, atm, of the osmagents may be greater than about zero atm, and generally about zero atm to about 500 atm, or higher.

The swellable, expandable polymer of the osmotically expandable driving layer, in addition to providing a driving source for delivering the active agent from the dosage form, may also function as a supporting matrix for an osmotically effective compound. The osmotic compound can be homogeneously or heterogeneously blended with the polymer to yield the desired expandable wall or expandable pocket. The composition in a presently preferred embodiment comprises (a) at least one polymer and at least one osmotic compound, or (b) at least one solid osmotic compound. Generally, a composition will comprise about 20% to about 90% by weight of polymer and about 80% to about 10% by weight of osmotic compound, with a presently preferred composition comprising about 35% to about 75% by weight of polymer and about 65% to about 25% by weight of osmotic compound.

The active agent of the osmotic pump dosage form may be formulated as a thermo-responsive formulation in which the active agent is dispersed in a thermo-responsive composition. Alternatively, the osmotic pump dosage form may contain a thermo-responsive element comprising a thermo-responsive composition at the interface of the osmotic push layer and the active agent composition. Representative thermo-responsive compositions and their melting points are as follows: Cocoa butter (32° C.-34° C.), cocoa butter plus 2% beeswax (35° C.-37° C.), propylene glycol monostearate and distearate (32° C.-35° C.), hydrogenated oils such as hydrogenated vegetable oil (36° C.-37.5° C.), 80% hydrogenated vegetable oil and 20% sorbitan monopalmitate (39° C.-39.5° C.), 80% hydrogenated vegetable oil and 20% polysorbate 60, (36° C.-37° C.), 77.5% hydrogenated vegetable oil, 20% sorbitan trioleate, 2.5% beeswax and 5.0% distilled water, (37° C.-38° C.), mono-, di-, and triglycerides of acids having from 8-22 carbon atoms including saturated and unsaturated acids such as palmitic, stearic, oleic, lineolic, linolenic and archidonic; triglycerides of saturated fatty acids with mono- and diglycerides (34° C.-35.5° C.), propylene glycol mono- and distearates 3(33° C.-34° C.), partially hydrogenated cottonseed oil (35° C.-39° C.), a block polymer of polyoxy-alkylene and propylene glycol; block polymers comprising 1,2-butylene oxide to which is added ethylene oxide; block copolymers of propylene oxide and ethylene oxide, hardened fatty alcohols and fats (33° C.-36° C.), hexadienol and hydrous lanolin triethanolamine glyceryl monostearate (38° C.), eutectic mixtures of mono-, di-, and triglycerides (35° C.-39° C.), WITEPSOL#15, triglyceride of saturated vegetable fatty acid with monoglycerides (33.5° C.-35.5° C.), WITEPSOL H32 free of hydroxyl groups (31° C.-33° C.), WITEPSOL W25 having a saponification value of 225-240 and a melting point of (33.5° C.-35.5° C.), WITEPSOL E75 having a saponification value of 220-230 and a melting point of (37° C.-39° C.), a polyalkylene glycol such as polyethylene glycol 1000, a linear polymer of ethylene oxide (38° C.-41° C.), polyethylene glycol 1500 (38° C.-41° C.), polyethylene glycol monostearate (39° C.-42.5° C.), 33% polyethylene glycol 1500, 47% polyethylene glycol 6000 and 20% distilled water (39° C.-41° C.), 30% polyethylene glycol 1500, 40% polyethylene glycol 4000 and 30% polyethylene glycol 400, (33° C.-38° C.), mixture of mono-, di-, and triglycerides of saturated fatty acids having 11 to 17 carbon atoms, (33° C.-35° C.), and the like. The thermo-responsive compositions, including thermo-responsive carriers are useful for storing the active agent in a solid composition at a temperature of about 20° C. to about 33° C., maintaining an immiscible boundary at the swelling composition interface, and for dispensing the agent in a flowable composition at a temperature greater than about 33° C. and preferably between about about 33° C. and about 40° C.

The amount of active agent present in the osmotic pump dosage form is about 10 mg to about 2 g or more. The osmotic dosage form may be formulated for once daily or less frequent administration.

The active agent of the osmotic pump dosage form may be formulated by a number of techniques known in the art for formulating solid and liquid oral dosage forms. The active agent of the osmotic pump dosage form may be formulated by wet granulation. In an exemplary wet granulation method, the active agent and the ingredients comprising the active agent layer are blended using an organic solvent, such as isopropyl alcohol-ethylene dichloride 80:20 v:v (volume:volume) as the granulation fluid. Other granulating fluid such as denatured alcohol 100% may be used for this purpose. The ingredients forming the active agent layer are individually passed through a screen such as a 40-mesh screen and then thoroughly blended in a mixer. Next, other ingredients comprising the active agent layer are dissolved in a portion of the granulation fluid, such as the cosolvent described above. Then the latter prepared wet blend is slowly added to the active agent blend with continual mixing in the blender. The granulating fluid is added until a wet blend is produced, which wet mass then is forced through a screen such as a 20-mesh screen onto oven trays. The blend is dried for about 18 to about 24 hours at about 30° C. to about 50° C. The dry granules are sized then with a screen such as a 20-mesh screen. Next, a lubricant is passed through a screen such as an 80-mesh screen and added to the dry screen granule blend. The granulation is put into milling jars and mixed on a jar mill for about 1 to about 15 minutes. The push layer may also be made by the same wet granulation techniques. The compositions are pressed into their individual layers in a KILIAN press-layer press.

Another manufacturing process that can be used for providing the active agent layer and osmotically expandable driving layer comprises blending the powered ingredients for each layer independently in a fluid bed granulator. After the powered ingredients are dry blended in the granulator, a granulating fluid, for example, poly(vinyl-pyrrolidone) in water, or in denatured alcohol, or in 95:5 ethyl alcohol/water, or in blends of ethanol and water is sprayed onto the powders. Optionally, the ingredients can be dissolved or suspended in the granulating fluid. The coated powders are then dried in a granulator. This process granulates the ingredients present therein while adding the granulating fluid. After the granules are dried, a lubricant such as stearic acid or magnesium stearate is added to the granulator. The granules for each separate layer are pressed then in the manner described above.

The active agent formulation and osmotic push layer of the osmotic dosage form may also be manufactured by mixing an active agent with composition forming ingredients and pressing the composition into a solid lamina possessing dimensions that correspond to the internal dimensions of the compartment. In another manufacture, the active agent and other active agent composition-forming ingredients and a solvent are mixed into a solid, or a semisolid, by methods such as ballmilling, calendaring, stirring or rollmilling, and then pressed into a preselected layer forming shape. Next, a layer of a composition comprising an osmopolymer and an optional osmagent are placed in contact with the layer comprising the active agent. The layering of the first layer comprising the active agent and the second layer comprising the osmopolymer and optional osmagent composition can be accomplished by using a conventional layer press technique. The semipermeable wall can be applied by molding, spraying or dipping the pressed bilayer's shapes into wall forming materials. An air suspension coating procedure which includes suspending and tumbling the two layers in current of air until the wall forming composition surrounds the layers is also used to form the semi-permeable wall of the osmotic dosage forms.

The dispenser of the osmotic pump dosage form may be in the form of a capsule. The capsule may comprise an osmotic hard capsule and/or an osmotic soft capsule. The osmotic hard capsule may be composed of two parts, a cap and a body, which are fitted together after the larger body is filled with the active agent. The osmotic hard capsule may be fitted together by slipping or telescoping the cap section over the body section, thus completely surrounding and encapsulating the active agent. Hard capsules may be made by techniques known in the art.

The soft capsule of the osmotic pump dosage form may be a one-piece osmotic soft capsule. Generally, the osmotic soft capsule is of sealed construction encapsulating the active agent. The soft capsule may be made by various processes, such as the plate process, the rotary die process, the reciprocating die process, and the continuous process.

Materials useful for forming the capsule of the osmotic pump dosage form are commercially available materials including gelatin, gelatin having a viscosity of about 5 to about 30 millipoises and a bloom strength up to about 150 grams; gelatin having a bloom value of about 160 to about 250; a composition comprising gelatin, glycerine, water and titanium dioxide; a composition comprising gelatin, erythrosin, iron oxide and titanium dioxide; a composition comprising gelatin, glycerine, sorbitol, potassium sorbate and titanium dioxide; a composition comprising gelatin, acacia, glycerin, and water; and the like, and combinations comprising one or more of the foregoing materials.

The semipermeable wall forming composition can be applied to the exterior surface of the capsule in laminar arrangement by molding, forming, air spraying, dipping or brushing with a semipermeable wall forming composition. Other techniques that can be used for applying the semipermeable wall are the air suspension procedure and the pan coating procedures. The air suspension procedure includes suspending and tumbling the capsule arrangement in a current of air and a semipermeable wall forming composition until the wall surrounds and coats the capsule. The procedure can be repeated with a different semipermeable wall forming composition to form a semipermeable laminated wall.

Exemplary solvents suitable for manufacturing the semipermeable wall include inert inorganic and organic solvents that do not adversely harm the materials, the capsule wall, the active agent, the thermo-responsive composition, the expandable member, or the final dispenser. Solvents for manufacturing the semipermeable wall may be aqueous solvents, alcohols, ketones, esters, ethers, aliphatic hydrocarbons, halogenated solvents, cycloaliphatics, aromatics, heterocyclic solvents, and combinations comprising one or more of the foregoing solvents. Particular solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride, nitroethane, nitropropane, tetrachloroethane, ethyl ether, isopropyl ether, cyclohexane, cyclooctane, benzene, toluene, naphtha, 1,4-dioxane, tetrahydrofuran, water, and mixtures thereof such as acetone and water, acetone and methanol, acetone and ethyl alcohol, methylene dichloride and methanol, and ethylene dichloride,methanol, and combinations comprising one or more of the foregoing solvents. The semipermeable wall may be applied at a temperature a few degrees less than the melting point of the thermo-responsive composition. Alternatively, the thermo-responsive composition can be loaded into the dispenser after applying the semipermeable wall.

The exit means or hole in the osmotic pump dosage form, for releasing the active agent, can be formed by mechanical or laser drilling, or by eroding an erodible element in the wall, such as a gelatin plug. The orifice can be a polymer inserted into the semipermeable wall, which polymer is a porous polymer and has at least one pore, or which polymer is a microporous polymer and has at least one micropore.

Solid State Dispersions

Another dosage form is a solid state dispersion. A “solid state dispersion” is a dispersion of one or more active agents in an inert carrier or matrix in a solid state prepared by a melting (fusion), solvent, or combined melt-solvent method. The dispersion of an active ingredient in a solid carrier or diluent by traditional mechanical mixing is not included within the definition of this term. Solid state dispersions are particularly advantageous for use with poorly soluble drugs.

Suitable carriers include, for example, hydroxypropyl cellulose, methyl cellulose, carboxymethyl cellulose, sodium carboxymethyl cellulose, cellulose acetate phthalate, cellulose acetate butyrate, hydroxyethyl cellulose, ethyl cellulose, polyvinyl alcohol, polypropylene, dextrans, dextrins, hydroxypropyl-beta-cyclodextrin, chitosan, co(lactic/glycolid) copolymers, poly(orthoester), poly(anhydrate), polyvinyl chloride, polyvinyl acetate, ethylene vinyl acetate, lectins, carbopols, silicon elastomers, polyacrylic polymers, maltodextrins, lactose, fructose, inositol, trehalose, maltose, raffinose, polyvinylpyrrolidone (PVP), polyethylene glycol (PEG), and alpha-, beta-, and gamma-cyclodextrins, and combinations comprising one or more of the foregoing carriers.

Suitable methods for forming solid state dispersions include, for example, the “solvent method”, in which the active ingredient is conventionally dispersed in a water soluble carrier by dissolving a physical mixture containing the active ingredient and the pharmaceutically acceptable carrier in a common organic solvent and then removing the solvent by evaporation. The resulting solid dispersion is recovered and used in the preparation of suitable pharmaceutical compositions. Manufacture of solid dispersions by the fusion or “melt” process involves combination of the pharmaceutically acceptable carrier and the poorly water soluble drug where the two components are allowed to melt at temperatures at or above the melting point of both the drug and the carrier. In the fusion process, the drug and carrier are first physically mixed and then both are melted. The molten mixture is then cooled rapidly to provide a congealed mass which is subsequently milled to produce a powder.

Another method for forming a solid dispersion comprises a solvent process comprising forming a solution comprising a carrier and a non-aqueous solvent. Suitable non-aqueous solvents include, for example, an alcohol selected from methanol, ethanol, n-propanol, iso-propanol, n-butanol, iso-butanol, and sec-butanol, and combinations comprising one or more of the foregoing solvents. The non-aqueous solvent may be dry or anhydrous. In forming a solution of a polymeric carrier and a non-aqueous solvent, it is understood that heating of the solution is allowable, but is not required, provided that the temperature does not result in decomposition or degradation of any materials.

Upon forming the solution, the process proceeds by dissolving the free base of a poorly water soluble active agent in the solution thus formed. Heating is allowed, but not required. Addition of a poorly soluble drug is not limited to one drug but might encompass a combination of one or more drugs provided at least one drug is a poorly water soluble drug in the form of a free base. The ratio by weight of carrier to poorly soluble drug can be about 5:1 to about 1:1; preferably about 4:1 to about 1:1; more preferably about 3:1 to about 1.5:1; most preferably about 2:1. The order of addition for the polymeric carrier, the nonaqueous solvent and the free base of the poorly water soluble drug is interchangeable. For example, the free base drug could be dissolved into the non-aqueous solvent after which the polymeric carrier is added.

Upon dissolution of the free base drug, the process proceeds converting the free base of the active agent to a pharmaceutically acceptable salt. The salt can be formed by addition of an inorganic or an organic acid which preferably is non-toxic and pharmaceutically acceptable. The acid may be added either as a gas, a liquid or as a solid dissolved into a nonaqueous solvent. The acid may be dry hydrogen chloride and the molar quantity of acid added to the solution of the active agent free base and carrier may either be in stoichiometric proportion to the active agent free base or be in excess of the molar quantity of the active agent free base, especially when added as a gas. Upon addition of the acid, the formed free base salt remains dissolved in solution with the polymeric carrier.

Lastly, upon formation of the free base salt, the process proceeds by recovering the non-aqueous solvent to form a solid state dispersion of the free base salt in the polymeric carrier. A method of removal of the non-aqueous solvent which renders a substantially homogeneous solid state dispersion is intended. Suitable methods of evaporation under vacuum include rotoevaporation, static vacuum drying, and a combination thereof. One skilled in the art of pharmaceutical formulations can determine a reasonable temperature at which the non-aqueous solvent can be removed, provided the temperature is not so high as to cause degradation or decomposition of the materials; however, such as about 20° C. to about 50° C. Evaporation of the non-aqueous solvent should render a solid state dispersion which is homogeneous and substantially free of non-aqueous solvent. By substantially free it is meant that the solid state dispersion contains less than about 20% by weight of residual non-aqueous solvent, preferably less than about 10%, more preferably less then about 5%, most preferably less then about 1%.

The ratio of active agent free base to the pharmaceutically acceptable carrier can be varied over a wide range and depends on the concentration of active agent required in the pharmaceutical dosage form ultimately administered. However, the preferred range of active agent in the solid dispersion is about 16% to about 50% of the total solid dispersion weight, more preferable is about 20% to about 50%, even more preferable is about 25% to about 40%, most preferable is about 33% of the total dispersion weight.

Alternatively, the general method for preparation of a solid dispersion can proceed by a fusion process wherein a carrier is mixed with a poorly water soluble drug, or drug combination, to form an intimate mixture. The mixture is heated at or near the temperature of the highest melting point of either the pharmaceutically acceptable carrier or poorly water soluble drug or drug combination, thus forming a melt. The polymeric carrier may be polyethylene glycol. A preferred ratio by weight of water soluble pharmaceutically acceptable polymeric carrier to poorly water soluble drug about 5:1 to about 1:1; preferably about 4:1 to about 1:1; more preferably about 3:1 to about 1.5:1; most preferably about 2:1.

Upon forming the molten homogeneous melt, the process proceeds by diffusing dry hydrogen chloride gas through the molten drug/carrier mixture to effect salt formation of the drug. Lastly, upon formation of the free base salt, the process proceeds by cooling the molten homogeneous melt by conventional methods to form a water soluble solid state dispersion.

Controlled-Release Formulations for Release Into the Stomach and Upper Gastrointestinal Tract

An exemplary controlled-release formulation is one in which a formulation in which the active agent is dispersed in a polymeric matrix that is water-swellable rather than merely hydrophilic, that has an erosion rate that is substantially slower than its swelling rate, and that releases the active agent primarily by diffusion. The rate of diffusion of the active agent out of the matrix can be slowed by increasing the active agent particle size, by the choice of polymer used in the matrix, and/or by the choice of molecular weight of the polymer. The matrix is a relatively high molecular weight polymer that swells upon ingestion, preferably to a size that is at least about twice its unswelled volume, and that promotes gastric retention during the fed mode. Upon swelling, the matrix may also convert over a prolonged period of time from a glassy polymer to a polymer that is rubbery in consistency, or from a crystalline polymer to a rubbery one. The penetrating fluid then causes release of the active agent in a gradual and prolonged manner by the process of solution diffusion, i.e., dissolution of the active agent in the penetrating fluid and diffusion of the dissolved drug back out of the matrix. The matrix itself is solid prior to administration and, once administered, remains undissolved in (i.e., is not eroded by) the gastric fluid for a period of time sufficient to permit substantially all of the active agent to be released by the solution diffusion process during the fed mode. By substantially all, it is meant greater than or equal to about 90 wt %, preferably greater than or equal to about 95 wt % of the active agent or pharmaceutically acceptable salt thereof is released. The rate-limiting factor in the release of the active agent may be therefore controlled diffusion of the active agent from the matrix rather than erosion, dissolving or chemical decomposition of the matrix.

For highly soluble active agents, the swelling of the polymeric matrix thus achieves two objectives—(i) the tablet swells to a size large enough to cause it to be retained in the stomach during the fed mode, and (ii) it retards the rate of diffusion of the highly soluble active agent long enough to provide multi-hour, controlled delivery of the active agent into the stomach.

The water-swellable polymer forming the matrix is a polymer that is non-toxic, that swells in a dimensionally unrestricted manner upon imbibition of water, and that provides for sustained-release of an incorporated active agent. Examples of suitable polymers include, for example, cellulose polymers and their derivatives (such as for example, hydroxyethylcellulose, hydroxypropylcellulose, carboxymethylcellulose, and microcrystalline cellulose, polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, poly (2-ethyl-2-oxazoline), poly(ethyleneimine), polyurethane hydrogels, and crosslinked polyacrylic acids and their derivatives. Further examples are copolymers of the polymers listed in the preceding sentence, including block copolymers and grafted polymers. Specific examples of copolymers are PLURONIC® and TECTONIC®, which are polyethylene oxide-polypropylene oxide block copolymers available from BASF Corporation, Chemicals Div., Wyandotte, Mich., USA.

The terms “cellulose” and “cellulosic” denote a linear polymer of anhydroglucose. Cellulosic polymers include, for example, alkyl-substituted cellulosic polymers that ultimately dissolve in the gastrointestinal (GI) tract in a predictably delayed manner. Alkyl-substituted cellulose derivatives may be those substituted with alkyl groups of 1 to 3 carbon atoms each. Specific examples are methylcellulose, hydroxymethylcellulose, hydroxyethylcellulose, hydroxypropylcellulose, hydroxypropylmethylcellulose, and carboxymethylcellulose. In terms of their viscosities, one class of suitable alkyl-substituted celluloses includes those whose viscosity is about 100 to about 110,000 centipoise as a 2% aqueous solution at 20° C. Another class includes those whose viscosity is about 1,000 to about 4,000 centipoise as a 1% aqueous solution at 20° C. Exemplary alkyl-substituted celluloses are hydroxyethylcellulose and hydroxypropylmethylcellulose. A specific example of a hydroxyethylcellulose is NATRASOL® 250HX NF (National Formulary), available from Aqualon Company, Wilmington, Del., USA.

Suitable polyalkylene oxides are those having the properties described above for alkyl-substituted cellulose polymers. An example of a polyalkylene oxide is poly(ethylene oxide), which term is used herein to denote a linear polymer of unsubstituted ethylene oxide. Poly(ethylene oxide) polymers having molecular weights of about 4,000,000 and higher are preferred. More preferred are those with molecular weights of about 4, 500,000 to about 10,000,000, and even more preferred are polymers with molecular weights of about 5,000,000 to about 8,000,000. Preferred poly(ethylene oxide)s are those with a weight-average molecular weight within the range of about 1×10⁵ to about 1×10⁷, and preferably within the range of about 9×10⁵ to about 8×10⁶. Poly(ethylene oxide)s are often characterized by their viscosity in solution. A preferred viscosity is about 50 to about 2,000,000 centipoise for a 2% aqueous solution at 20° C. Two specific example of poly(ethylene oxide)s are POLYOX® NF, grade WSR Coagulant, molecular weight 5 million, and grade WSR 303, molecular weight 7 million, both available from Dow.

Polysaccharide gums, both natural and modified (semi-synthetic) can be used. Examples are dextran, xanthan gum, gellan gum, welan gum and rhamsan gum.

Crosslinked polyacrylic acids of greatest utility are those whose properties are the same as those described above for alkyl-substituted cellulose and polyalkylene oxide polymers. Preferred crosslinked polyacrylic acids are those with a viscosity of about 4,000 to about 40,000 centipoise for a 1% aqueous solution at 25° C. Three specific examples are CARBOPOL® NF grades 971P, 974P and 934P (BFGoodrich Co., Specialty Polymers and Chemicals Div., Cleveland, Ohio, USA). Further examples are polymers known as WATER LOCK®, which are starch/acrylates/acrylamide copolymers available from Grain Processing Corporation, Muscatine, Iowa, USA.

The hydrophilicity and water swellability of these polymers cause the active agent-containing matrices to swell in size in the gastric cavity due to ingress of water in order to achieve a size that will be retained in the stomach when introduced during the fed mode. These qualities also cause the matrices to become slippery, which provides resistance to peristalsis and further promotes their retention in the stomach. The release rate of an active agent from the matrix is primarily dependent upon the rate of water imbibition and the rate at which the active agent dissolves and diffuses from the swollen polymer, which in turn is related to the solubility and dissolution rate of the active agent, the active agent particle size and the active agent concentration in the matrix. Also, because these polymers dissolve very slowly in gastric fluid, the matrix maintains its physical integrity over at least a substantial period of time, in many cases at least 90% and preferably over 100% of the dosing period. The particles will then slowly dissolve or decompose. Complete dissolution or decomposition may not occur until 24 hours or more after the intended dosing period ceases, although in most cases, complete dissolution or decomposition will occur within 10 to 24 hours after the dosing period.

The dosage forms may include additives that impart a small degree of hydrophobic character, to further retard the release rate of the active agent into the gastric fluid. One example of such a release rate retardant is glyceryl monostearate. Other examples are fatty acids and salts of fatty acids, one example of which is sodium myristate. The quantities of these additives when present can vary; and in most cases, the weight ratio of additive to active agent will be about 1:20 to about 1:1, and preferably about 1:8 to about 1:2.

The amount of polymer relative to the active agent can vary, depending on the active agent release rate desired and on the polymer, its molecular weight, and excipients that may be present in the formulation. The amount of polymer will be sufficient however to retain at least about 40% of the active agent within the matrix one hour after ingestion (or immersion in the gastric fluid). Preferably, the amount of polymer is such that at least 50% of the active agent remains in the matrix one hour after ingestion. More preferably, at least 60%, and most preferably at least 80%, of the active agent remains in the matrix one hour after ingestion. In all cases, however, the active agent will be substantially all released from the matrix within about ten hours, and preferably within about eight hours, after ingestion, and the polymeric matrix will remain substantially intact until all of the active agent is released. The term “substantially intact” is used herein to denote a polymeric matrix in which the polymer portion substantially retains its size and shape without deterioration due to becoming solubilized in the gastric fluid or due to breakage into fragments or small particles.

The water-swellable polymers can be used individually or in combination. Certain combinations will often provide a more controlled-release of the active agent than their components when used individually. An examplary combination is cellulose-based polymers combined with gums, such as hydroxyethyl cellulose or hydroxypropyl cellulose combined with xanthan gum. Another example is poly(ethylene oxide) combined with xanthan gum.

The benefits of this dosage form will be achieved over a wide range of active agent loadings, with the weight ratio of active agent to polymer of 0.01:99.99 to about 80:20. Preferred loadings (expressed in terms of the weight percent of active agent relative to total of active agent and polymer) are about 15% to about 80%, more preferably about 30% to about 80%, and most preferably in certain cases about 30% to about 70%. For certain applications, however, the benefits will be obtained with active agent loadings of 0.01% to 80%, and preferably 15% to 80%.

As indicated above, the dosage forms find their greatest utility when administered to a subject who is in the digestive state (also referred to as the postprandial or “fed” mode). The postprandial mode is distinguishable from the interdigestive (or “fasting”) mode by their distinct patterns of gastroduodenal motor activity, which determine the gastric retention or gastric transit time of the stomach contents.

In the interdigestive mode, the fasted stomach exhibits a cyclic activity called the interdigestive migrating motor complex (IMMC). The cyclic activity occurs in four phases:
Phase I is the most quiescent, lasts 45 to 60 minutes, and develops few or no contractions.
Phase II is marked by the incidence of irregular intermittent sweeping contractions that gradually increase in magnitude.
Phase III, which lasts 5 to 15 minutes, is marked by the appearance of intense bursts of peristaltic waves involving both the stomach and the small bowel.
Phase IV is a transition period of decreasing activity which lasts until the next cycle begins.

The total cycle time is approximately 90 minutes, and thus, powerful peristaltic waves sweep out the contents of the stomach every 90 minutes during the interdigestive mode. The IMMC may function as an intestinal housekeeper, sweeping swallowed saliva, gastric secretions, and debris to the small intestine and colon, preparing the upper tract for the next meal while preventing bacterial overgrowth. Pancreatic exocrine secretion of pancreatic peptide and motilin also cycle in synchrony with these motor patterns.

Combination

In addition to the embodiments where galantamine is the only active agent, the invention includes combination dosage forms that also contain other active agents useful in the treatment of conditions such as conditions such as dementia, especially Alzheimer's dementia. Other embodiments include galantamine in combination with other active agents to treat acute cholinergic effects that may occur.

The invention includes formulations comprising combinations of galantamine, a galantamine salt, and an additional active agent such as a cognition enhancer, an anti-emetic, a proton-pump inhibitor, or an antacid. The invention also includes combination dosage forms that contain one or more cognitive enhancer as the additional active agent. Such combinations are useful for treating both the psychosis and memory deficits of Alzheimer's dementia.

Suitable cognition enhancers include, for example, memantine, metrifonate, rivastigmine, tacrine, a combination comprising at least one of the foregoing cognition enhancers, and the like. When combined in a dosage formulation with galantamine, the cognition enhancer may be present in the formulation at about 1 to about 99 weight percent based on the total of galantamine and cognition enhancer. Within this range, the cognition enhancer may be present at about 10 to about 80 weight percent, preferably about 20 to about 60 weight percent, and yet more preferably about 30 to about 45 weight percent based on the total of galantamine and cognition enhancer.

As many patients experience gastric disturbances when taking galantamine, due to the cholinergic effects of the active agent, a formulation comprising a combination of galantamine and an anti-emetic, a proton-pump inhibitor, or an antacid would provide a means to minimized the effects. A suitable anti-emetic includes, for example, dolasetron mesylate, ondansetron, metoclopramide, granisetron, prochlorperazine, and the like.

The invention includes combination dosage forms in which an antacid is included in the invention. Suitable antacids for use in the combination include, acid neutralizers, such as aluminum hydroxide, magnesium hydroxide, aluminum carbonate, calcium carbonate, sodium bicarbonate, or a combination comprising at least one of the foregoing; histamine-2 antagonists (H2-antagonists) examples of which include cimetidine, famotidine, nizatidine, ranitidine; and proton pump inhibitors, such as omeprazole, esomeprazole magnesium, lansoprazole, esomeprazole, pantoprazole, rabeprazole, or a combination comprising at least one of the foregoing proton pump inhibitors.

Dissolution Profiles for Galantamine Dosage Forms

The invention provides the active agent dosage forms and dosage forms comprising galantamine and one or more other active agent described herein formulated so that particular dissolution profiles are achieved.

In one embodiment the invention provides a dosage form that exhibits a dissolution profile that is substantially identical to that of REMINYL in the same dissolution media.

In another embodiment, a fast dissolve galantamine formulation, described above, comprising a pharmaceutically acceptable carrier, wherein the carrier is substantially free of a spray dried mixture of lactose monohydrate and microcrystalline cellulose exhibits a dissolution profile such that after 0.5 hour at least about 80% of the galantamine or galantamine salt is released after combining the dosage form with 500 ml of purified water at 37° C. in Apparatus 2 (USP, <711 > Dissolution, paddle, 50 rpm).

In yet another embodiment, a dosage formulation comprises galantamine or a pharmaceutically acceptable salt thereof, and a pharmaceutically acceptable carrier; and wherein the formulation exhibits a dissolution profile such that after 0.5 hour less than about 75% of the galantamine or galantamine salt is released after combining the dosage form with 500 ml of purified water at 37° C. in Apparatus 2 (USP, <711 > Dissolution, paddle, 50 rpm). Suitable carriers described above may be used to prepare a formulation having the described dissolution profile.

In another embodiment, a sustained-release formulation comprises the active agent and a release-retarding material such that the formulation exhibits a dissolution profile such that less than about 18% of the galantamine or galantamine salt is released in 1 hour, and less than about 80% of the galantamine or galantamine salt is released in 10 hours after combining the formulation with USP buffer pH 6.8 at 37° C. in an Apparatus 2 (USP, <711> Dissolution, paddle, 50 rpm).

In yet another embodiment, a dosage formulation comprises a pharmaceutically effective amount of galantamine or a pharmaceutically acceptable salt thereof; and a pharmaceutically acceptable excipient, wherein the dosage formulation exhibits a dissolution profile such that less than about 18% of the galantamine or galantamine salt is released in 1 hour, and less than about 80% of the galantamine or galantamine salt is released in 10 hours after combining the dosage form with USP buffer pH 6.8 at 37° C. in an Apparatus 2 (USP, <711> Dissolution, paddle, 50 rpm).

In a further embodiment, a dosage formulation comprising galantamine or a pharmaceutically acceptable salt thereof; and a pharmaceutically acceptable excipient exhibits a dissolution profile such that after 1 hour from 1 to about 18% of the galantamine or galantamine salt is released, after 2 hours about 15 to about 35% of the galantamine or galantamine salt is released, after 3 hours about 30 to about 50% of the galantamine or galantamine salt is released, and after 4 hours about 50 to about 70% of the galantamine or galantamine salt is released in 10 hours after combining the dosage form with USP buffer pH 6.8 at 37° C. in an Apparatus 2 (USP, <711> Dissolution, paddle, 50 rpm.

Yet another embodiment includes a dosage formulation comprising a pharmaceutically effective amount of galantamine or a pharmaceutically acceptable salt thereof; and a pharmaceutically acceptable excipient wherein the dosage formulation exhibits a dissolution profile such that after 10 hours less than about 80% of the galantamine or galantamine salt is released after combining the dosage form with USP buffer pH 6.8 at 37° C. in an Apparatus 2 (USP, <71 1> Dissolution, paddle, 50 rpm).

Another embodiment includes a dosage formulation comprising a pharmaceutically effective amount of galantamine or a pharmaceutically acceptable salt thereof; and a pharmaceutically acceptable excipient, wherein the dosage formulation exhibits a dissolution profile such that after 1 hour about 5 to about 15% of the galantamine or galantamine salt is released, after 2 hours about 10 to about 25% of galantamine or galantamine salt is released,after 4 hours about 15 to about 35% of the galantamine or galantamine salt is released, and after 8 hours about 25 to about 50% of galantamine or galantamine salt is released in 10 hours after combining the dosage form with USP buffer pH 6.8 at 37° C. in an Apparatus 2 (USP, <711> Dissolution, paddle, 50 rpm.

Pharmacokinetic Properties of Galantamine Dosage Forms

The invention provides the active agent dosage forms and dosage forms comprising active agent and one or more other active agent (combinations) described herein formulated so that particular plasma levels, Cₘₐₓ, Tₘₐₓ, and AUC values are achieved.

In one embodiment the invention provides a dosage form that exhibits a Cₘₐₓ value and AUC from time of administration to 24 hours after administration that are from 80% to 120% of the Cₘₐₓ value and AUC from time of administration to 24 hours after administration exhibited by REMINYL under the same conditions. Also provided herein is a dosage form exhibits a Cₘₐₓ value and AUC from time of administration to 36 hours after administration that are from 80% to 120% of the Cₘₐₓ value and AUC from time of administration to 36 hours after administration exhibited by REMINYL under the same conditions.

In another embodiment, a oral dosage formulation comprises galantamine or a pharmaceutically acceptable salt thereof in controlled-release form, wherein the formulation provides a first maximum plasma concentration of the galantamine (Cₘₐₓ₁) between 0 hours and about 12 hours after administration, and a second maximum plasma concentration of the galantamine (Cₘₐₓ₂) between about 12 hours and about 24 hours after administration at steady-state.

Within this embodiment, the formulation provides a first maximum galantamine plasma concentration (Cₘₐₓ₁) between 0 hours and about 12 hours after administration, a second maximum galantamine plasma concentration (Cₘₐₓ₂) between about 12 hours and about 24 hours after administration, and an galantamine plasma concentration at about 24 hours after administration (C₂₄), wherein the average galantamine plasma concentration between about Cₘₐₓ₁, and about Cₘₐₓ₂ is substantially equal to the average galantamine plasma concentration between about Cₘₐₓ₂ and about C₂₄.

Furthermore, the formulation of this embodiment provides a first maximum galantamine plasma concentration (Cₘₐₓ₁) and a first minimum galantamine plasma concentration (Cₘᵢₙ₁) between 0 hours and about 12 hours after administration, a second maximum galantamine plasma concentration (Cₘₐₓ₂), and a galantamine plasma concentration at about 24 hours after administration (C₂₄), wherein the ratio of Cₘₐₓ₁ to Cₘᵢₙ₁ is less than about 4:1, preferably less than about 2:1, or the ratio of Cₘₐₓ₂ to C₂₄ is less than about 4:1, preferably less than about 3:1.

Also within this embodiment, the Cₘₐₓ₂ occurs about 12 to about 14 hours after administration. Furthermore, at steady state the difference between the ratio of Cₘₐₓ₁ to Cₘᵢₙ₁ and the ratio of Cₘₐₓ₂ to C₂₄ is less than about 50%, preferably less than about 40%, and more preferably less than about 30%.

The galantamine formulations comprised herein preferably provide a mean maximum plasma concentration of galantamine from about 10 to about 60 ng/ml and a mean minimum plasma concentration from about 3 to about 15 ng/ml after repeated administration every day through steady-state conditions.

Manufacture of Dosage Forms

Amorphous Technology

Amorphous solids consist of disordered arrangements of molecules and do not possess a distinguishable crystal lattice. Galantamine may be prepared in such a way that substantially all of the active agent is present in amorphous form.

A process for preparing solid, amorphous galantamine comprises mixing active agent free base or a pharmaceutically acceptable salt thereof with a solvent, such as water, and a pharmaceutically acceptable polymeric carrier; and drying to form a composition comprising amorphous active agent and polymeric carrier.

In another aspect, a pharmaceutical composition comprises active agent salt in amorphous, solid form, and polymeric carrier, prepared by the aforementioned process.

Suitable pharmaceutically acceptable polymeric carriers include, for example, hydroxypropyl cellulose, methyl cellulose, carboxymethyl cellulose, sodium carboxymethyl cellulose, cellulose acetate phthalate, cellulose acetate butyrate, hydroxyethyl cellulose, ethyl cellulose, polyvinyl alcohol, polypropylene, dextrans, dextrins, hydroxypropyl-beta-cyclodextrin, chitosan, co(lactic/glycolid) copolymers, poly(orthoester), poly(anhydrate), polyvinyl chloride, polyvinyl acetate, ethylene vinyl acetate, lectins, carbopols, silicon elastomers, polyacrylic polymers, maltodextrins, polyvinylpyrrolidone (PVP), polyethylene glycol (PEG), and alpha-, beta-, and gamma-cyclodextrins, and combinations comprising one or more of the foregoing carriers.

Preferred polymeric carriers are one or more of polyvinylpyrrolidone, hydroxypropylmethyl cellulose, hydroxypropyl cellulose, methyl cellulose, block co-polymers of ethylene oxide and propylene oxide, and polyethylene glycol, wherein a more preferred polymeric carrier is polyvinylpyrrolidone (PVP) having an average molecular weight of about 2,500 to about 3,000,000. A most preferred polymeric carrier is polyvinylpyrrolidone having an average molecular weight of from about 10,000 to about 450,000.

The polymeric carrier is preferably miscible with both the active agent free base and the salt, capable of keeping the salt in a homogeneous noncrystalline solid state dispersion after the solvent has been removed by evaporation and chemically inert with respect to the free base of the active ingredient, the salt of the free base, and the acid solution.

The active agent may be added in either free base or salt form. When the active agent is added in free base form, the process comprises adding an acid corresponding to a pharmaceutically acceptable salt of the active agent to the mixture or solution of the free base. The free base is then converted to a salt in situ, for example by addition of an inorganic or an organic acid. The acid may be added either as a gas, a liquid or as a solid dissolved into the solvent. A preferred acid is hydrogen bromide and the molar quantity of acid added to the solution of active agent free base and carrier may either be in stoichiometric proportion to the active agent free base or be in excess of the molar quantity of the active agent free base, especially when added as a gas.

The preferred range of acid added is about 1.0 to about 1.8 times the molar quantity of galantamine free base. Preferred molar ratios of active agent to hydrogen bromide are about 1:1 to 1:1.8, more preferably about 1:1.1. Although hydrogen bromide may be added as a gas, the preferred method to add the hydrogen bromide is in the form of hydrogen bromide dissolved into a solvent. It is understood that upon addition of the acid, the formed free base salt remains dissolved in solution with the polymeric carrier.

Galantamine, polymeric carrier, and solvent may be combined in any order. It is preferred that they be combined in a manner so as to form a solution of active agent salt and the polymeric carrier.

In forming a solution of polymeric carrier and solvent, heating of the solution is not necessary at lower concentrations but is strongly preferred at higher concentrations, provided that the temperature does not result in decomposition or degradation of any materials. It is preferred to add the active agent free base or active agent salt after dissolving the polymeric carrier in the solvent, suitably at about 25° to about 100° C., preferably at about 45° to about 80° C. When the active agent is added as a free base, it is preferred to form a salt at a temperature at which the final solution is clear. For the most preferred embodiments, a temperature of at least about 60° C. may result in a clear solution of the active agent salt being formed, although for other concentrations and embodiments, clear solutions are formed at other temperatures. It is preferred to only add enough heat to form a clear solution.

The ratio of active agent to the polymeric carrier can be varied over a wide range and depends on the concentration of active agent required in the pharmaceutical dosage form ultimately administered. The ratio by weight of polymeric carrier to active agent salt is about 20:1 to about 0.5:1; preferably about 4:1 to about 1:1; more preferably about 3:1 to about 1.5:1; most preferably about 2:1.

Preferably a clear solution is formed. Upon formation of the clear solution, the process proceeds by recovering the solvent to form a solid state dispersion of the free base salt in the polymeric carrier. Any method of removal of the solvent which renders a homogeneous solid state dispersion is intended, although preferred are methods of evaporation under vacuum or spray drying. Methods of evaporation under vacuum include rotary evaporation, static vacuum drying and the combination thereof. It is understood that one skilled in the art of pharmaceutical formulations can determine a reasonable temperature at which the solvent can be removed, provided the temperature is not so high as to cause degradation or decomposition of the materials; however, it is preferred that evaporation occurs at about 25° C. to about 100° C. Evaporation of the solvent should render a solid state dispersion which is homogeneous and substantially free of solvent. By substantially free it is meant that the solid state dispersion contains less than 20% by weight of residual solvent, preferably less than 10%, more preferably less than 5%, most preferably less than 1%.

The ratio of galantamine free base to the polymeric carrier can be varied over a wide range and depends on the concentration of active agent required in the pharmaceutical dosage form ultimately administered. However, the preferred range of active agent in the solid dispersion is about 10% to about 50% of the total solid dispersion weight, more preferable is about 20% to about 50%, even more preferable is about 25% to about 40%, most preferable is about 33% of the total dispersion weight. In terms of weight ratio of polymeric carrier to active agent, a preferred range is about 0.4:1 to 20:1.

Suitable pharmaceutically acceptable excipients can be added in the process. Examples of pharmaceutically acceptable excipients include diluents, binders, disintegrants, coloring agents, flavoring agents, lubricants and/or preservatives. The pharmaceutical composition may be formulated by conventional methods of admixture such as blending, filling, granulation and compressing. These agents may be utilized in conventional manner.

Optional Additional Additives

Excipients

Excipients are components added to active agent pharmaceutical formulation other than the active agent. Excipients may be added to facilitate manufacture, enhance stability, control release, enhance product characteristics, enhance bioavailability, enhance patient acceptability, etc. Pharmaceutical excipients include binders, disintegrants, lubricants, glidants, compression aids, colors, sweeteners, preservatives, suspending agents, dispersing agents, film formers, flavors, printing inks, etc. Binders hold the ingredients in the dosage form together. Exemplary binders include, for example, polyvinyl pyrrolidone, hydroxypropyl cellulose, hydroxypropyl methylcellulose, methylcellulose and hydroxyethyl cellulose, sugars, and combinations comprising one or more of the foregoing binders. Disintegrants expand when wet causing a tablet to break apart. Exemplary disintegrants include water swellable substances, for example, low-substituted hydroxypropyl cellulose, e.g. L-HPC; cross-linked polyvinyl pyrrolidone (PVP-XL), e.g. Kollidon® CL and Polyplasdone® XL; cross-linked sodium carboxymethylcellulose, e.g. Ac-di-sol®, Primellose®; sodium starch glycolate, e.g. Primojel®; sodium carboxymethylcellulose, e.g. Nymcel ZSB10®; sodium carboxymethyl starch, e.g. Explotab®; ion-exchange resins, e.g. Dowex® or Amberlite®; microcrystalline cellulose, e.g. Avicel®; starches and pregelatinized starch, e.g. Starch 1500®, Sepistab ST200 ®; formalin-casein, e.g. Plas-Vita®, and combinations comprising one or more of the foregoing water swellable substances. Lubricants, for example, aid in the processing of powder materials. Exemplary lubricants include calcium stearate, glycerol behenate, magnesium stearate, mineral oil, polyethylene glycol, sodium stearyl fumarate, stearic acid, talc, vegetable oil, zinc stearate, and combinations comprising one or more of the foregoing lubricants. Glidants include, for example, silicon dioxide.

Fillers

Certain dosage forms described herein contain a filler, such as a water insoluble filler, water soluble filler, and combinations thereof. The filler may be a water insoluble filler, such as silicon dioxide, titanium dioxide, talc, alumina, starch, kaolin, polacrilin potassium, powdered cellulose, microcrystalline cellulose, and combinations comprising one or more of the foregoing fillers. Exemplary water-soluble fillers include water soluble sugars and sugar alcohols, preferably lactose, glucose, fructose, sucrose, mannose, dextrose, galactose, the corresponding sugar alcohols and other sugar alcohols, such as mannitol, sorbitol, xylitol, and combinations comprising one or more of the foregoing fillers.

Preparation of the Active Agent

Preparation of Subunits

The active agent and any optional additives may be prepared in many different ways, for example as subunits. Pellets comprising an active ingredient can be prepared, for example, by a melt pelletization technique. In this technique, the active ingredient in finely divided form is combined with a binder and other optional inert ingredients, and thereafter the mixture is pelletized, e.g., by mechanically working the mixture in a high shear mixer to form the pellets (e.g., pellets, granules, spheres, beads, etc., collectively referred to herein as “pellets”). Thereafter, the pellets can be sieved in order to obtain pellets of the requisite size. The binder material may also be in particulate form and has a melting point above about 40° C. Suitable binder substances include, for example, hydrogenated castor oil, hydrogenated vegetable oil, other hydrogenated fats, fatty alcohols, fatty acid esters, fatty acid glycerides, and the like, and combinations comprising one or more of the foregoing binders.

Oral dosage forms may be prepared to include an effective amount of melt-extruded subunits containing the active agent or other active agents in the form of multiparticles within a capsule. For example, a plurality of the melt-extruded muliparticulates can be placed in a gelatin capsule in an amount sufficient to provide an effective release dose when ingested and contacting by gastric fluid.

Subunits, e.g., in the form of multiparticulates, can be compressed into an oral tablet using conventional tableting equipment using standard techniques. The tablet formulation may include excipients such as, for example, an inert diluent such as lactose, granulating and disintegrating agents such as cornstarch, biding agents such as starch, and lubricating agents such as magnesium stearate.

Alternatively, the subunits containing the active agent and optionally containing additional active agents are added during the extrusion process and the extrudate can be shaped into tablets by methods know in the art. The diameter of the extruder aperture or exit port can also be adjusted to vary the thickness of the extruded strands. Furthermore, the exit part of the extruder need not be round; it can be oblong, rectangular, etc. The exiting strands can be reduced to particles using a hot wire cutter, guillotine, etc.

A melt-extruded multiparticulate system can be, for example, in the form of granules, spheroids, pellets, or the like, depending upon the extruder exit orifice. The terms “melt-extruded multiparticulate(s)” and “melt-extruded multiparticulate system(s)” and “melt-extruded particles” are used interchangeably herein and include a plurality of subunits, preferably within a range of similar size and/or shape. The melt-extruded multiparticulates can be about 0.1 to about 12 mm in length and have a diameter of about 0.1 to about 5 mm. In addition, the melt-extruded multiparticulates can be any geometrical shape within this size range. Alternatively, the extrudate can simply be cut into desired lengths and divided into unit doses of the therapeutically active agent without the need of a spheronization step.

The melt-extruded dosage forms can further include combinations of melt-extruded multiparticulates containing one or more of the therapeutically active agents before being encapsulated. Furthermore, the dosage forms can also include an amount of the active agent formulated for immediate-release for prompt therapeutic effect. The active agent formulated for immediate-release can be incorporated or coated on the surface of the subunits after preparation of the dosage forms (e.g., controlled-release coating or matrix-based). The dosage forms can also contain a combination of controlled-release beads and matrix multiparticulates to achieve a desired effect.

A melt-extruded material may be prepared without the inclusion of subunits containing the active agent, which are added thereafter to the extrudate. Such formulations have the subunits and other active agents blended together with the extruded matrix material. The mixture is then tableted in order to provide release of the active agent or other active agents. Such formulations can be particularly advantageous, for example, when an active agent included in the formulation is sensitive to temperatures needed for softening the hydrophobic material and/or the retardant material.

The oral dosage form containing the active agent may be in the form of micro-tablets enclosed inside a capsule, e.g. a gelatin capsule. For this, a gelatin capsule as is employed in pharmaceutical formulations can be used, such as the hard gelatin capsule known as CAPSUGEL, available from Pfizer.

Particles

Many of the oral dosage forms described herein contain the active agent and optionally additional active agents in the form of particles. Such particles may be compressed into a tablet, present in a core element of a coated dosage form, such as a taste masked dosage form, a press coated dosage form, or an enteric coated dosage form, or may be contained in a capsule, osmotic pump dosage form, or other dosage form.

For particles, such as powder particles, present in the core element of a coated dosage form, the core element may have a particle size distribution with a median of about 100 μm. The particles in the distribution may vary from about 1 μm to about 250 μm, more preferably from 25 μm to about 250 μm, most preferably about 35 μm to about 125 μm. If the median of the distribution is close to either extreme of the distribution, the taste masking or sustained-release characteristics may be affected. In a particle size range of about 25 μm to about 250 μm, no more than about 25% of particles can be less than about 25 μm, and no more than about 25% can be over about 250 μm.

Another parameter to consider is particle shape. Particle shape can influence the coverage and stability of the coat. Both the crystallinity of the active agent and the aspect ratio of the particles are related to particle shape. It is preferred that the active agent of the coated dosage forms has a crystalline morphology, however, sharp angles on a crystal can cause weaknesses in the coat. These sharp corners may lead to stress points on the coat and cause weaknesses in the structure possibly leading to premature release of the active agent from the dosage form. Furthermore, areas of thin coating are susceptible to breaking and cracking and hence ineffective for sustained-release and taste masking.

Regarding the aspect ratio, a low aspect ratio is preferred. The aspect ratio is a measure of the length to breadth. For example, a low aspect ratio of about 1 would be a box or sphere. Crystals with a high aspect ratio are more pointed with needle-like crystals. Crystals with a high aspect ratio may result in a relatively thin coat at the crystal needle tips leading to a more rapid release rate of the active agent than is preferred. A low aspect ratio spherical shape of the particle is advantageous for both solubility of the coat and high payload of the active agent. Therefore, it is most preferable that the aspect ratio is less than about 3, more preferably about 1 to about 2, and most preferably approximately 1 providing a substantially rounded shape.

Inconsistencies in size and shape can lead to inconsistent coating. Where the particles containing the active agent are of different size and shape, polymeric coating materials such as ethyl cellulose may deposit differently on each particle. It is therefore preferable for coated dosage forms that substantially all particles of the dosage form have substantially the same size and shape so that the coating process is better controlled and maintained.

Preparation of Dosage Forms

The term “dosage form” denotes a form of a formulation that contains an amount sufficient to achieve a therapeutic effect with a single administration. When the formulation is a tablet or capsule, the dosage form is usually one such tablet or capsule. The frequency of administration that will provide the most effective results in an efficient manner without overdosing will vary with the characteristics of the particular active agent, including both its pharmacological characteristics and its physical characteristics such as solubility, and with the characteristics of the swellable matrix such as its permeability, and the relative amounts of the drug and polymer. In most cases, the dosage form will be such that effective results will be achieved with administration no more frequently than once every eight hours or more, preferably once every twelve hours or more, and even more preferably once every twenty-four hours or more.

The dosage form can be prepared by various conventional mixing, comminution and fabrication techniques readily apparent to those skilled in the chemistry of drug formulations. Examples of such techniques are as follows:
(1) Direct compression, using appropriate punches and dies; the punches and dies are fitted to a suitable rotary tableting press;
(2) Injection or compression molding using suitable molds fitted to a compression unit
(3) Granulation followed by compression; and
(4) Extrusion in the form of a paste, into a mold or to an extrudate to be cut into lengths.

When particles are made by direct compression, the addition of lubricants may be helpful and sometimes important to promote powder flow and to prevent capping of the particle (breaking off of a portion of the particle) when the pressure is relieved. Useful lubricants are magnesium stearate (in a concentration of from 0.25% to 3% by weight, preferably less than 1% by weight, in the powder mix), and hydrogenated vegetable oil (preferably hydrogenated and refined triglycerides of stearic and palmitic acids at about 1% to 5% by weight, most preferably about 2% by weight. Additional excipients may be added to enhance powder flowability and reduce adherence.

Pellets in Capsules

Oral dosage forms may be prepared to include an effective amount of melt-extruded subunits in the form of multiparticles within a capsule. For example, a plurality of the melt-extruded muliparticulates can be placed in a gelatin capsule in an amount sufficient to provide an effective release dose when ingested and contacted by gastric fluid.

Pellets in Tablets

The subunits, e.g., in the form of multiparticulates, can be compressed into an oral tablet using conventional tableting equipment using standard techniques.

Tablets in Capsules

The composition may be in the form of micro-tablets enclosed inside a capsule, e.g. a gelatin capsule. For this, a gelatin capsule employed in the pharmaceutical formulation field can be used, such as the hard gelatin capsule known as CAPSUGEL, available from Pfizer.

Coatings

The formulations described herein may be coated with a functional or non-functional coating. The coating may comprise an amount more than 0 to about 40 weight percent of the composition. The coating material may include a polymer, preferably a film-forming polymer, for example, methyl cellulose, ethyl cellulose, hydroxypropyl cellulose, hydroxypropyl methyl cellulose, hydroxybutyl methyl cellulose, cellulose acetate, cellulose propionate, cellulose acetate propionate, cellulose acetate butyrate, cellulose acetate phthalate, carboxymethyl cellulose, cellulose triacetate, cellulose sulphate sodium salt, poly(methyl methacrylate), poly (ethyl methacrylate), poly (butyl methacrylate), poly (isobutyl methacrylate), poly (hexyl methacrylate), poly (phenyl methacrylate), poly (methyl acrylate), poly (isopropyl acrylate), poly (isobutyl acrylate), poly (octadecyl acrylate), poly (ethylene), poly (ethylene) low density, poly (ethylene)high density, (poly propylene), poly (ethylene glycol poly (ethylene oxide), poly (ethylene terephthalate), poly(vinyl alcohol), poly(vinyl isobutyl ether), poly(viny acetate), poly (vinyl chloride), polyvinyl pyrrolidone, and combinations comprising one or more of the foregoing polymers.

In applications such as taste-masking, the polymer can be a water-insoluble polymer. Water insoluble polymers include ethyl cellulose or dispersions of ethyl cellulose, acrylic and/or methacrylic ester polymers, cellulose acetates, butyrates or propionates or copolymers of acrylates or methacrylates having a low quaternary ammonium content, and the like, and combinations comprising one or more of the foregoing polymers.

In controlled-release applications, for example, the coating can be a hydrophobic polymer that modifies the release properties of the active agent from the formulation. Suitable hydrophobic or water insoluble polymers for controlled-release include, for example, methacrylic acid esters, ethyl cellulose, cellulose acetate, polyvinyl alcohol-maleic anhydride copolymers, β-pinene polymers, glyceryl esters of wood resins, and combinations comprising one or more of the foregoing polymers.

The inclusion of an effective amount of a plasticizer in the coating composition may improve the physical properties of the film. For example, because ethyl cellulose has a relatively high glass transition temperature and does not form flexible films under normal coating conditions, it may be advantageous to add plasticizer to the ethyl cellulose before using the same as a coating material. Generally, the amount of plasticizer included in a coating solution is based on the concentration of the polymer, e.g., most often from about 1 to about 50 percent by weight of the polymer. Concentrations of the plasticizer, however, can be determined by routine experimentation.

Examples of plasticizers for ethyl cellulose and other celluloses include plasticizers such as dibutyl sebacate, diethyl phthalate, triethyl citrate, tributyl citrate, triacetin, and combinations comprising one or more of the foregoing plasticizers, although it is possible that other water-insoluble plasticizers (such as acetylated monoglycerides, phthalate esters, castor oil, etc.) can be used.

Examples of plasticizers for acrylic polymers include citric acid esters such as triethyl citrate, tributyl citrate, dibutyl phthalate, 1,2-propylene glycol, polyethylene glycols, propylene glycol, diethyl phthalate, castor oil, triacetin, and combinations comprising one or more of the foregoing plasticizers, although it is possible that other plasticizers (such as acetylated monoglycerides, phthalate esters, castor oil, etc.) can be used.

An example of a functional coating comprises a coating agent comprising a poorly-water-permeable component (a) such as, an alkyl cellulose, for example an ethylcellulose, such as AQUACOAT (a 30% dispersion available from FMC, Philadelphia, Pa.) or SURELEASE (a 25% dispersion available from Colorcon, West Point, Pa.) and a water-soluble component (b), e.g., an agent that can form channels through the poorly-water-permeable component upon the hydration or dissolution of the soluble component. Preferably, the water-soluble component is a low molecular weight, polymeric material, e.g., a hydroxyalkylcellulose, hydroxyalkyl(alkylcellulose), and carboxymethylcellulose or salts thereof. Particular examples of these water soluble polymeric materials include hydroxyethylcellulose, hydroxypropylcellulose, hydroxyethylmethylcellulose, hydroxypropylmethylcellulose, carboxymethylcellulose, sodium carboxymethylcellulose, and combinations comprising one or more of the foregoing materials. The water-soluble component can comprise hydroxypropylmethylcellulose, such as METHOCEL (Dow). The water-soluble component is preferably of relatively low molecular weight, preferably less than or equal to about 25,000 molecular weight, or preferably less than or equal to about 21,000 molecular weight.

In the functional coating, the total of the water soluble portion (b) and poorly-water permeable portion (a) are present in weight ratios (b):(a) of about 1:4 to about 2:1, preferably about 1:2 to about 1:1, and more preferably in a ratio of about 2:3. While the ratios disclosed herein are preferred for duplicating target release rates of presently marketed dosage forms, other ratios can be used to modify the speed with which the coating permits release of the active agent. The functional coating may comprise about 1% to about 40%, preferably about 3% to about 30%, more preferably about 5% to about 25%, and yet more preferably about 6% to about 10% of the total formulation.

In certain embodiments, particularly where the coating provides taste masking, it is preferred that the coating is substantially continuous coat and substantially hole-free. By “substantially continuous coating” is meant a coating which retains a smooth and continuous appearance when magnified 1000 times under a scanning electron microscope and wherein no holes or breakage of the coating are evident.

Suitable methods can be used to apply the coating to the active agent. Processes such as simple or complex coacervation, interfacial polymerization, liquid drying, thermal and ionic gelation, spray drying, spray chilling, fluidized bed coating, pan coating, electrostatic deposition, may be used. A substantially continuous nature of the coating may be achieved, for example, by spray drying from a suspension or dispersion of the active agent in a solution of the coating composition including a polymer in a solvent in a drying gas having a low dew point.

When a solvent is used to apply the coating, the solvent is preferably an organic solvent that constitutes a good solvent for the coating material, but is substantially a non-solvent or poor solvent for of the active agent. While the active agent may partially dissolve in the solvent, it is preferred that the active ingredient will precipitate out of the solvent during the spray drying process more rapidly than the coating material. The solvent may be selected from alcohols such as methanol, ethanol, halogenated hydrocarbons such as dichloromethane (methylene chloride), hydrocarbons such as cyclohexane, and combinations comprising one or more of the foregoing solvents. Dichloromethane (methylene chloride) has been found to be particularly suitable.

The concentration of polymer in the solvent will normally be less than about 75% by weight, and typically about 10 to about 30% by weight. After coating, the coated dosage forms may be allowed to cure for at least about 1 to about 2 hours at a temperature of about 50° C. to about 60° C., more preferably of about 55° C.

The coatings may be about 0.005 micrometers to about 25 micrometers thick, preferably about 0.05 micrometers to about 5 micrometers.

### EXAMPLES

The following examples further illustrate the invention but, of course, should not be construed as in any way limiting its scope.

### Example 1

### Fast Dissolve Galantamine Hydrobromide Solid Dosage Form

A mixture of the components of Table 1 (three dosages A, B, and C) is intimately mixed using a planetary mixer. The mixture is then compressed into direct compression tablets.

TABLE 1

| | Amount (milligram) | | |
|---|---|---|---|
| Components | A | B | C |
| Galantamine | 5.126 | 10.252 | 15.378 |
| hydrobromide | | | |
| Lactose (anhydrous) | 176.324 | 171.324 | 166.072 |
| Microcrystalline cellulose | 55 | 55 | 55 |
| Croscarmellose sodium | 12 | 12 | 12 |
| Colloidal anhydrous silica | 0.35 | 0.35 | 0.35 |
| Magnesium stearate | 1.2 | 1.2 | 1.2 |
| Total weight | 250 | 250 | 250 |

Tablets prepared from the formula in Table 1 are tested for dissolution profiles using 500 milliliters purified water as the dissolution media at 37° C. in Apparatus 2 (USP 23, <711> Dissolution) using a paddle speed of 50 rotations per. minute (rpm).

The tablets from the formula in Table 1 are film coated with the component in Table 2 using a coating pan. The resulting film coated tablets are tested for dissolution profiles using the method described for the uncoated tablets.

TABLE 2

| Components | Amount (milligram) |
|---|---|
| Hypromellose 2910 | 5 |
| Talc | 2.5 |
| Macrogel 6000 | 0.9 |
| Purified water^{a} | 50 |
| Total weight | 3.9 |

^{a}Not present in final coated tablet

### Example 2

### Controlled-Release Formulation Containing Galantamine Hydrobromide

Controlled-release formulations of galantamine hydrobromide are prepared according to the following procedure and the formulations in Tables 3-4.

TABLE 3

| Components | Amount (milligram) |
|---|---|
| Galantamine hydrobromide | 25.0 |
| Lactose | 125.0 |
| Kollidon 90F (povidone USP) | 9.0 |
| Purified Water | 200.0^{a} |
| Stearic Acid | 3.2 |
| Total weight (dry) | 162.2 |

^{a}Not present in final tablet

Povidone is first dissolved in water. Galantamine hydrobromide is placed in the top spraying chamber of Glatt GPCG1 fluidized bed apparatus. The solution of povidone is sprayed onto the active ingredient, with an air flow of 100-110 m³/h, a liquid flow of 6-7 g/min, an inlet temperature of 65° C., and a spraying pressure of 2.8 bar.

Once the granulation is completed, granules are passed through a sieve (1 mm mesh) and stearic acid is weighed, added and blended in a V-blender. The resulting mixture is pressed into tablets ( 9/32 inch diameter). These tablet cores are then coated with the formulation of Table 4.

TABLE 4

| Components | Amount (milligram) |
|---|---|
| Ethocel PR100 (ethylcellulose) | 7.05 |
| Kollidon 90F (povidone USP) | 7.05 |
| PEG 1450 | 2.10 |
| Denatured alcohol^{a} | 210.00 |
| Total weight (dry) | 16.2 |

^{a}Not present in final coated tablet

Ethocel, povidone, and PEG 1450 are first dissolved in denatured alcohol. The coating solution is then sprayed onto the tablet cores in a coating pan (Vector LCDS), with spray parameters of airflow of 100-110 m³/h, liquid flow of 6-7 g/min, inlet temperature of 65° C., and a spraying pressure of 2.8 bar.

### Example 3

### Sustained-Release and Immediate-Release in a Single Formulation Containing Galantamine Hydrobromide.

A coating comprising galantamine hydrobromide is coated onto the coated tablet of Example 2 allowing for the immediate-release of galantamine and a controlled-release. Example 3 is prepared according to the following procedure and the formulations in Tables 5-7.

TABLE 5

| Components | Amount (milligram) |
|---|---|
| Galantamine hydrobromide | 25.0 |
| Lactose | 110.0 |
| Kollidon 90F (povidone USP) | 9.0 |
| Purified Water^{a} | 160.0 |
| Stearic Acid | 3.2 |
| Total weight (dry) | 147.2 |

^{a}Not present in final tablet

The preparation process is identical to the one of Example 2. These tablet cores are then coated with the following formulation in Table 6.

TABLE 6

| Components | Amount (milligram) |
|---|---|
| Ethocel PR100 (ethylcellulose) | 7.05 |
| Kollidon 90F (povidone USP) | 7.05 |
| PEG 1450 | 2.10 |
| Denatured alcohol^{a} | 210.00 |
| Total weight (dry) | 16.2 |

^{a}Not present in final coated tablet

The coating process is as in Example 2. A second coating, according to the formula in Table 7 is then sprayed onto the coated tablet.

TABLE 7

| Components | Amount (milligram) |
|---|---|
| Galantamine hydrobromide | 15.0 |
| Ethocel PR100 (ethylcellulose) | 5.0 |
| Denatured alcohol^{a} | 30.0 |
| Total weight (dry) | 16.2 |

^{a}Not present in final coated tablet

The coating process is furthered in a manner identical to the one for the first coat.

### Example 4

### Controlled-Release Formulation Containing Galantamine Hydrobromide

Controlled-release formulations of galantamine are prepared according to the following procedure and the formulations in Tables 8-9.

TABLE 8

| Components | Amount (milligram) |
|---|---|
| Galantamine hydrobromide | 25.0 |
| Lactose | 125.0 |
| Stearic Acid | 5.0 |
| Avicel (microcrystalline cellulose) | 20.0 |
| Total weight (dry) | 175.0 |

Galantamine hydrobromide and stearic acid are placed in the chamber of Glatt GPCG1 fluidized bed apparatus having the following parameters: air flow of 100-110 m³/h and an inlet temperature of 60-65 ° C. The powders are fluidized with hot air. The powders are heated until the product temperature reaches 50-55° C.; at this point granulation takes place. The product is then cooled to room temperature.

Once the granulation is completed, granules are passed through a sieve (1 mm) and microcrystalline cellulose is weighed, added and blended in a V-blender. The resulting mixture is pressed into tablets ( 9/32 inch diameter). These tablet cores are then coated with the formulation in Table 9.

TABLE 9

| Components | Amount (milligram) |
|---|---|
| Ethocel PR 100 (ethylcellulose) | 5.0 |
| Kollidon 90F (povidone USP) | 5.0 |
| PEG 1450 | 1.5 |
| Denatured alcohol^{a} | 210.0 |
| Total weight (dry) | 11.5 |

^{a}Not present in final coated tablet

The coating process for Example 2 was used.

### Example 5

### Sustained Formulation Containing Galantamine Hydrobromide

An extended-release formulation is prepared using the formulation in Table 10.

TABLE 10

| Components | Amount (milligram) |
|---|---|
| Galantamine hydrobromide | 25.0 |
| Lactose | 125.0 |
| PVA (Polyvinyl Acetate USP) | 5.30 |
| Purified Water^{a} | 110.0 |
| Glyceryl behenate | 4.7 |
| Total weight (dry) | 160.0 |

^{a}Not present in final coated tablet

PVA is first dissolved in water. Galantamine hydrobromide is placed in the top spraying chamber of Glatt GPCG1 fluidized bed apparatus. The solution of PVA is sprayed onto the active ingredient, with the parameters of air flow of 100-110 m³/h, liquid flow of 6-7 g/min, inlet temperature of 65° C., and spraying pressure of 2.8 bar.

Once the granulation is completed, granules are passed through a sieve (1 mm mesh) and glyceryl behenate is weighed, added and blended in a V-blender. The resulting mixture is pressed into tablets ( 9/32 inch diameter). These tablet cores are then coated with the formulation of Table 1 as a first coating.

TABLE 11

| Components | Amount (milligram) |
|---|---|
| Ethocel PR100 (ethylcellulose) | 7.0 |
| Kollidon 90F (povidone USP) | 3.0 |
| PEG 1450 | 1.5 |
| Denatured alcohol^{a} | 210.0 |
| Total weight (dry) | 11.5 |

^{a}Not present in final coated tablet

Ethocel, povidone and PEG 1450 are first dissolved in denatured alcohol. The coating solution is then sprayed onto the tablet cores in a coating pan (Vector LCDS), with the following spraying parameters: air flow of 100-110 m³/h, liquid flow of 6-7 g/min, inlet temperature of 65° C., and spraying pressure of 2.8 bar. The coated tablets are then coated with a second coating having the formula as found in Table 12.

TABLE 12

| Components | Amount (milligram) |
|---|---|
| Eudragit L30 D-55 | 7.0 |
| Silicon dioxide | 2.1 |
| PEG 1450 | 1.4 |
| Triethyl citrate | 0.7 |
| Water^{a} | 40.0 |
| Total weight (dry) | 11.5 |

^{a}Not present in final coated tablet

PEG and triethyl citrate 1450 are first dissolved in half the quantity of water. Eudragit is then added to the solution and stirred for 45 minutes. Silicon dioxide is suspended in the remaining quantity of water and is homogenized. The silicon dioxide suspension is then added to the Eudragit dispersion. The tablets are coated in a coating pan (Vector LCDS), with the following spraying parameters: air flow of 100-110 m³/h, liquid flow of 6-7 g/min, inlet temperature of 55° C., and spraying pressure of 2.8 bar.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. (canceled)

2. A dosage formulation, comprising:
galantamine or a pharmaceutically acceptable salt, hydrate, solvate, crystal form, diastereomer, prodrug, or mixture thereof; and
a pharmaceutically acceptable carrier; and
wherein the formulation exhibits a dissolution profile such that after 0.5 hour loss than about 75% of the galantamine or galantamine salt is released after combininig the dosage formulation with 500 ml or purified water at 37° C. in Apparatus 2 (USP, <711 > Dissolution, paddle, 50 rpm).

3. The formulation of claim 2, wherein the galantamine salt is galantamine hydrobromide.

4.-6. (canceled)

7. The formulation of claim 2, further comprising a cognition enhancer, an anti-emetic, a proton-pump inhibitor, an antacid, or a combination comprising at least one of the foregoing.

8. The formulation of claim 7, wherein the cognition enhancer is memantine, metrifonate, rivastigmine, tacrine, a pharmaceutically acceptable salt thereof or a combination comprising at least one of the foregoing cognition enhancers.

9. The formulation of claim 7, wherein the anti-emetic is dolasetron mesylate, ondansetron, metoclopramide, granisetron, prochlorperazine, a pharmaceutically acceptable salt thereof, or a combination comprising at least one of the foregoing anti-emetics.

10. The formulation of claim 7, wherein the antacid is aluminum hydroxide, magnesium, hydroxide, aluminum carbonate, calcium carbonate, sodium bicarbonate, or a combination comprising at least one of the foregoing antacids.

11. The formulation of claim 7, wherein the proton pump inhibitor is omeprazole, esomeprazole magnesium, lansoprazole, esomeprazole, pantoprazole, rabeprazole, or a combination comprising at least one of the foregoing proton pump inhibitors.

12. (canceled)

13. The formulation of claim 2, wherein the formulation provides bioequivalence according to FDA guidelines or criteria.

14. (canceled)

15. A sustained-release formulation, comprising:
galantamine or a pharmaceutically acceptable salt, hydrate, solvate, crystal form, diastereomer, prodrug, or mixture thereof; and
a release-retarding material, wherein the release-retarding material is all acrylate polymer, wax, modified cellulose, shellac, zein, hydrogenated vegetable oil, hydrogenated castor oil, or combinations comprising at least one of the foregoing release-retarding materials,
wherein the formulation exhibits a dissolution profile such that less than about 18% of the galantamine is released in 1 hour, and less than about 80% of the galantamine is released in 10 hours after combining the formulation with a dissolution medium at 37° C. in Apparatus 2 (USP, <711> Dissolution, paddle, 50 rpm).

16. The sustained-release formulation of claim 15, wherein the salt is galantamine hydrobromide.

17. The sustained-release formulation of claim 15, wherein the acrylate polymer is a methyl methacrylate copolymer, an ethoxyethyl methacrylate, a cyanoethyl methacrylate, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate, a polymethacrylate, a poly(methyl methacrylate) copolymer, a polyacrylamide, an aminoalkyl methacrylate copolymer, a glycidyl methacrylate copolymer, an ammonio methacrylate copolymer, or a combination comprising at least one of the foregoing acrylate polymers.

18. The sustained-release formulation of claim 15, wherein the modified cellulose is an alkyl cellulose, a hydroxyalkyl cellulose, or a combination comprising at least one of the foregoing modified celluloses.

19. The sustained-release formulation of claim 15, wherein the alkyl cellulose is methyl cellulose, ethyl cellulose, or a combination comprising at least one of the foregoing alkyl celluloses.

20. The sustained-release formulation of claim 15, wherein the hydroxyalkyl cellulose is hydroxyethyl cellulose, hydroxypropyl cellulose, hydroxypropylmethyl cellulose, hydroxypropylethylcellulose, hydroxypropylpropylcellulose, hydroxypropylbutylcellulose or a combination comprising at least one of the foregoing hydroxyalkyl celluloses.

21. The sustained-release formulation of claim 15, wherein the release-retarding material is in the form of a coating.

22. The sustained-release formulation of claim 21, wherein the coating comprises a water insoluble polymer.

23. The sustained-release formulation of claim 21, wherein the coating comprises an aqueous dispersion of a water insoluble polymer.

24.-38. (canceled)

39. The dosage formulation of claim 15,
wherein the dosage formulation exhibits a dissolution profile such that
after 1 hour about 5 to about 15% of the galantamine or galantamine salt is released,
after 2 hours about 10 to about 25% of galantamine or galantamine salt is released,
after 4 hours about 15 to about 35% of the galantamine or galantainine salt is released, and
after 8 hours about 25 to about 50% of galantamine or galantamine salt is released.

40.-50. (canceled)

51. The dosage formulation of claim 15, wherein the dissolution medium is 500 ml of purified water.

52. The dosage formulation of claim 15, wherein the dissolution medium is 500 ml of an aqueous buffer solution (USP, p11 4.5); an aqueous buffer solution (USP, p11 6.8); an aqueous buffer solution (USP, pH 7.5); or 0.1N HCl.
